(19) United States
(12) Patent Application Publication
(10) Pub. No.: US 2022/0053743 A1
(43) Pub. Date: Feb. 24, 2022

(54) SYSTEM AND METHOD FOR MOSQUITO PUPAE SORTING, COUNTING AND PACKAGING

(71) Applicant: Senecio Ltd., Kfar-Saba (IL)

(72) Inventors: Hanan LEPEK, Kfar-Saba (IL); Hasdi MATARASSO, Pardes Hana (IL); Tamir LEV, Haifa (IL)

(73) Assignee: Senecio Ltd., Kfar-Saba (IL)

(21) Appl. No.: 17/415,137

(22) PCT Filed: Dec. 17, 2019
(86) PCT No.: PCT/IL2019/051379

§ 371 Date: Jun. 17, 2021

Related U.S. Application Data
(60) Provisional application No. 62/785,514, filed on Dec. 27, 2018.
(60) Provisional application No. 62/780,767, filed on Dec. 17, 2018.

(51) Int. Cl. A01K 67/033; B03B 5/56

(52) U.S. Cl. CPC A01K 67/033; B03B 5/56

(57) ABSTRACT

A device for sorting insects into two or more classes based on size, the device comprising a drum with openings along the circumference. The insects are introduced to the drum at a first end, and the openings are sized to allow a first class of the insects to exit the drum through the circumference and the openings while the second class is retained to travel to far end of the drum, thereby sorting the insects into the classes. The drum may be held at a tilt to enable flow within the drum. The drum may rotate and a built in helix or helical screw may assist with flow. Water may be poured from above or from inside to flush insects through the openings or flush stuck insects back into the interior.

### RELATED APPLICATIONS

This application claims the benefit of priority from U.S. Provisional Patent Applications No. 62/780,767 filed on 17 Dec. 2018 and 62/785,514 filed 27 Dec. 2018, the contents of which are incorporated herein by reference in their entirety.

### FIELD AND BACKGROUND OF THE INVENTION

The present invention, in some embodiments thereof, relates to a system and method for mosquito pupae sorting, counting and/or packaging and, more particularly, but not exclusively, to a system that does not use a sieve.

Mosquito larvae are artificially grown in trays and personnel are required to check for development into pupae. If so, they currently manually separate the pupae from the larvae in a tedious process. Larvae return to the tray and the pupae are removed before they can emerge and fly away.

The processes of rearing the larvae, feeding them and then the process of loading pupae into release cartridges are disconnected from each other. Also the process of sex separation (if such is required) is disconnected from the larvae rearing process.

As the requirement for effective SIT projects requires the rearing and sorting of millions of mosquitoes, the above process has to be automated.

Mosquitoes can be sorted by their gender during their pupal stage to an accuracy of 97%-99% using sieving devices and such manual products are well used in the industry.

However, current available methods are limited in their throughput and ability to provide continuous operation and are only able to carry out sex sorting of relatively small numbers of pupae per unit time.

Patent application publication numbers US2018/0271073 A1, and US2018/0271072 A1, entitled “Sieving Device for Pupae Separation” together disclose a system for sorting pupa by having them sieved through a mesh which can move up and down.

Patent application number WO2013140167A1 entitled “Sorting apparatus for arthropods and method of use thereof”, also describes a sieving device for sorting of pupae.

U.S. Pat. No. 10,251,380 B2 to Sobecki et al, entitled Sieving Apparatus for Pupae Separation, discloses a sieving method for separating insect pupae in which a sieving apparatus is actuated to cycle between two elevations to cyclically submerge a sieve surface in a liquid so as to separate a pupa population with respect to size.

Colonization and Mass Rearing: Learning from Others in Malaria Journal 2009, 8 (Suppl 2): S4 Mark Q Benedict et al, considers as a conceptual idea a separator made of a rotating drum structure having gaps for sorting between larvae and pupae—see FIG. 1.

### SUMMARY OF THE INVENTION

The present embodiments may link these processes into a continuous cyclic operation and enable a repetitive and continuous sorting of male and female pupae, maximizing the number of healthy male pupae that are successfully reared and sorted through the system and made available for large scale SIT projects.

According to an aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being at a tilt such that the first end is higher than the second end to enable flow from the first end to the second end.

According to a further aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum further comprising a helix shape in the interior to cause flow through the interior as the helix is rotated.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being mounted and motorized for rotating, the drum being located below water outlets, the outlets being configured to pour water on the openings as the openings rotate past the water outlets to flush out insects stuck in the openings.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being mounted and motorized for rotating, the drum being located against a bank of mechanical protrusions, the mechanical protrusions being configured to extend into the openings as the openings rotate past the mechanical protrusions to clean out insects stuck in the openings.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being mounted and motorized for rotating, the openings comprising gaps between a plurality of discs, the discs comprising a first class of discs interspersed with a second class of discs, the first class of discs extending further into the interior than the second class of discs.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being mounted and motorized for rotating, the drum comprising an internal pipe for injecting water into the interior to flush the openings.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being mounted and motorized for rotating, the device comprising at least one collection container underneath the drum to collect the insects exiting through the circumference.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum comprising a funnel underneath the drum and a collection chamber, the funnel leading to the collection chamber, to collect the insects exiting through the circumference, and an indicator located at the collection chamber to provide an indication when the collection chamber contains a pre-defined number of insects.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the openings comprising gaps between discs the discs having an inner extent, the drum being mounted for raising and lowering with respect to an external water level between a first position wherein inner extents of the discs at a lower side of the drum are submerged and an second position in which at least the drum internal surface is above water level.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum comprising a helical screw rotatably mounted to rotate relative to the drum to propel insects and water from the first end to the second end.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the circumference being divided into at least two sections, each section having openings of different sizes respectively.

According to another aspect of some embodiments of the present invention there is provided a device for sorting insects into two or more classes based on size, the device comprising a drum, the drum having an interior being closed circumferentially and openings defined along the circumference, and the insects being introduced to the drum at a first end, the openings being sized to allow a first class of the insects to exit the drum through the circumference while the second class is retained to travel to a second end, thereby sorting the insects into the at least two classes, the drum being open at a second end opposite the first end, to allow the second class to flow out of the second end.

Devices may comprise a rotating screw inserted into the drum.

Devices may comprise a helical shape built onto an interior of the circumference.

In an embodiment, there are two classes, being male and female pupae, the male and female pupae being of different sizes and the openings being of a size selected to allow male pupae to pass through but to inhibit female pupae.

In an embodiment, there are three classes, larvae, male pupae and female pupae, the drum comprising two sections, a first section with openings sized for larvae, and a second section with openings sized for male pupae.

In an embodiment, there are four classes, larvae, small male pupae, large male pupae and female pupae, the drum comprising three sections, a first section with openings sized for larvae, a second section with openings sized for small male pupae and a third section with openings sized for large male pupae.

An input at the first end of the drum receives a mixture of water, larvae and male and female pupae, the input comprising a pourer for pouring the mixture into the drum.

The pourer may pour out the mixture into the interior in a plane of the first end. Alternatively, the pourer may pour the mixture into the interior over a length of the drum.

The drum may be a stationary mounted half drum.

The drum may be rotatably mounted.

Mechanical projections, which may for example be in the form of prongs, knives or teeth, for cleaning the openings to prevent clogging may be mounted to a frame via a tensioned mount.

Openings may comprise cuts in a cylinder or slots or gaps between discs or rings.

In the case that the openings are gaps between discs, a size of each opening may be defined by a spacer between respective discs.

At least some of the discs may have a longitudinal cross section comprising a convex outer edge and a flat interior. Some of the discs may have a longitudinal cross section that is flat overall without any convex part.

Drainage collection underneath the drum and a pump to recirculate water from the drainage for reuse in the drum may be provided.

The drum may be mounted such that an angle of tilt is adjustable.

In embodiments the rate of pouring of water and insects at the input is adjustable to optimize sorting.

According to a further aspect of the present invention there is provided apparatus for continuous insect rearing comprising:
a first circular production line rearing larvae to a pupa stage;
a second circular production line rearing pupae to an adult stage;
and a pupa unloading station configured to unload pupae from the first production line onto the second production line.

In an embodiment, the pupa unloading station comprises a device for sorting the pupae into male and female pupae, thereby to supply sex-sorted pupae to the second circular production line.

In an embodiment, the first circular production line comprises larva breeding trays containing larva in water, and the second production line comprises pupa breeding trays. The production lines may be synchronized to provide pupa breeding trays to empty the larva breeding trays into waiting pupa trays.

According to a yet further aspect of some embodiments of the present invention there is provided method of sorting pupae into at least a first class and a second class based on size, comprising:
Introducing pupae into a drum having openings large enough for pupae of a smaller of the classes to pass through but too small for pupae of a larger class;
Causing water to flow through the drum to carry the pupae past the openings;
Flushing water through the openings from within the drum to carry out the pupae of the smaller class for collection below the drum; and
Flushing water from outside the drum downwardly onto the drum to flush back into the drum pupae of the larger class that are stuck in the openings.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a simplified schematic view of a drum according to embodiments of the present invention;

FIG. 2 is a view of the drum of FIG. 1 from a different angle;

FIG. 3 is a simplified view of the wall of the drum of FIG. 1;

FIG. 4 is a side perspective view of the drum of FIG. 1;

FIG. 5 is a schematic cross-sectional view of three slots in the wall of the drum of FIG. 1;

FIGS. 6A-6D are schematic views showing pupae being flushed through or clogging the openings between the slots of FIG. 5;

FIG. 7 is a detail of a variation of the slots of FIG. 5;

FIG. 8 is a simplified block diagram of a control system for the drum of FIG. 1;

FIG. 9 is an end-on view of the drum of FIG. 1 with collecting containers;

FIG. 10 is a view from above of the drum of FIG. 9;

FIGS. 11A and 11B are views of a collection funnel and collection chamber of the drum of FIG. 1;

FIG. 12 is a view of a drainage arrangement for the collecting containers of FIG. 9;

FIGS. 13, 14 and 15 are views of alternative arrangements of slots forming the wall of the drum of FIG. 1;

FIG. 16 is a view of variable size netting used with the drum of FIG. 1;

FIG. 17 is a variation of the drum of FIG. 1 in which openings in the drum are provided between a series of tubes;

FIGS. 18 and 19 are two views showing how counting of pupae exiting from the drum of FIG. 1 may be carried out;

FIGS. 20 and 21 illustrate a screw integrally built into the drum of FIG. 1;

FIGS. 22 and 23 are schematic views of a helix built into the drum of FIG. 1;

FIG. 24 is a schematic diagram showing water and pupa flow through the drum of FIG. 1;

FIGS. 25A and 25B are details showing structures of slots to facilitate flow of pupae to reach the slots of the drum of FIG. 1;

FIG. 26 is a schematic illustration of a variation of the drum of FIG. 1 in which four classes of pupa and larvae are sorted;

FIG. 27 is a schematic illustration of the drum of FIG. 1 with reuse of water;

FIGS. 28-34 are a series of views of a device using the drum of FIG. 1 as an integrated sorting system for pupae (and larvae);

FIGS. 35 and 36 are simplified schematic illustrations of a mechanical element and a flexible mounting for declogging the openings of the drum of FIG. 1;

FIG. 37 is a block diagram of a controller and interface for operating the device of FIGS. 28-34;

FIG. 38 is a flow chart showing an exemplary sequence for operating the device of FIGS. 28-34;

FIGS. 39A and 39B are two diagrams showing water levels around a device according to claims 28 to 34 when operated using the sequence of FIG. 38;

FIG. 40 is a diagram showing the drum of FIGS. 39A and 39B in an upper position;

FIG. 41 is a detail showing the mounting of the declogging element of FIGS. 35 and 36 mounted around a drum according to embodiments of the present invention;

FIG. 42 is a schematic detail of a variation of a slot opening of the drum of FIG. 1;

FIGS. 43 and 44 are details of an embodiment according to the present invention in which the drum is a half cylinder;

FIG. 45 is a view of a cyclical production line for growing larvae according to embodiments of the present invention;

FIGS. 46 and 47 are details of the production line of FIG. 45;

FIG. 48 is a flow chart illustrating synchronized operation of a larva production line in conjunction with a pupa production line and showing an interaction therebetween according to embodiments of the present invention;

FIG. 49 is a time chart in stages of producing larvae and pupae according to embodiments of the present invention;

FIG. 50 is a simplified diagram of a larva production circle connected to a pupa production circle according to embodiments of the present invention; and

FIG. 51 is a simplified diagram of larva and pupae production integrated into a single production circle according to embodiments of the present invention.

### DESCRIPTION OF SPECIFIC EMBODIMENTS OF THE INVENTION

The present invention, in some embodiments thereof, relates to a system and method for mosquito pupae sorting, counting and/or packaging.

Embodiments may provide a device for sorting insects into two or more classes based on size, the device comprising a drum with openings along the circumference. The term “drum” refers to a cylinder shape where the outer wall may be continuous with openings or the outer wall may comprise rings or discs with gaps in between. In the case of a rotatable drum, discs constituting the cylindrical wall of the drum may rotate together. The insects are introduced to the drum at a first end, and the openings are sized to allow a first class of the insects to exit the drum through the circumference and the openings while the second class is retained to travel to far end of the drum, thereby sorting the insects into the classes.

Embodiments may provide a continuous flow process from the egg or larvae stage until the mosquitoes are packed into release boxes. The process may either be fully automated or semi-automated.

The drum may be held at a tilt to enable flow within the drum. The drum may rotate and a built in helix or helical screw may assist with flow. Water may be poured from above or from inside to flush insects through the openings or flush stuck insects back into the interior. Alternatively or additionally the drum may be located against a bank of protrusions, teeth or knives, which extend into the openings as the openings rotate past the teeth to clean out insects stuck in said openings and thus reduce clogging.

In an embodiment, the drum openings are gaps between discs. The discs may be identical or they may be made up of a first class of discs interspersed with a second class of discs, the first class of discs extending further into the interior than the second class of discs, with the effect of setting up channels between the deeper extending discs to slow down water flow and get more of the pupae to be channeled to the openings.

The drum may have an internal pipe that pours water into the drum to provide internal flow to carry the pupae and flush them through the openings when they fit.

The drum may have collection containers located below. These may be positioned to collect the insects that pass through the openings on the circumference of the drum. Others may be positioned at the end to collect the insects or pupae that do not pass through the circumference. The containers may be motorized so that they can be moved into position automatically and taken away when full and detectors may be used to determine when they are full. Such detection may be used for coordination operation of the drum so that sorting only occurs when a container is present.

In an embodiment, the drum may be mounted for raising and lowering with respect to an external water level between a lower position wherein inner extents of the discs at a lower side of the drum are submerged and an upper position in which said drum is above said water level. The lower position allows for the insect mixture to flow through the drum and the upper position allows for drainage.

The drum may include a helical screw to drive the water insect mixture through the drum. The drum may have two or more sections where the openings in the circumference are of different sizes so that sorting can be into more than two classes. In an embodiment, within each section there may be progression in the sizes of the openings. The drum may be open at the far end so that the largest class simply flows out of the opening. A container may be placed in position to collect the insects of the largest class as they flow out of the far end.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

An object of the present embodiments is to enable a repetitive and continuous sorting of male and female pupae, maximizing the number of healthy male pupae that are successfully sorted through the system.

According to one embodiment, the solution includes: a software controlled automated sorting device; the software controls actuating moving elements automatically based on feedback received from feedback sensors associated with the moving elements. The moving elements may include a rotating drum mounted at an angle. The rotating drum may consist of a number of tapered discs connected together and separated from each other by small spacers creating spacings between the discs, and a helical screw integrated within the drum and extending from side to side. A single liquid inlet plane area may pour insects into the drum. Other, water, inlets may pour water on top of the rotating drum, and a water pipe may extend internally from side to side along the drum, pouring water. A funnel and counting device may count the sorted males as they fall, and a collection area collects males beneath the tapered discs. A second collection area may be provided for the collection of females, and a drive system may propagate male containers and female containers as they are filled. A controller may synchronize and control operation of the water flow above and within the drum, and the rate of pouring of the pupae into the drum, to be synchronized with propagation of the collection containers as they are filled and in accordance with reading from the counting device.

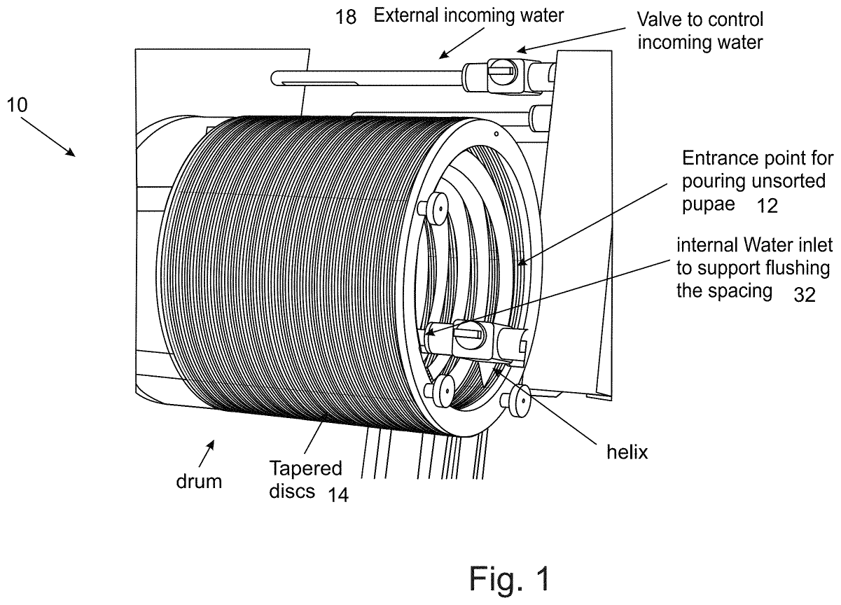

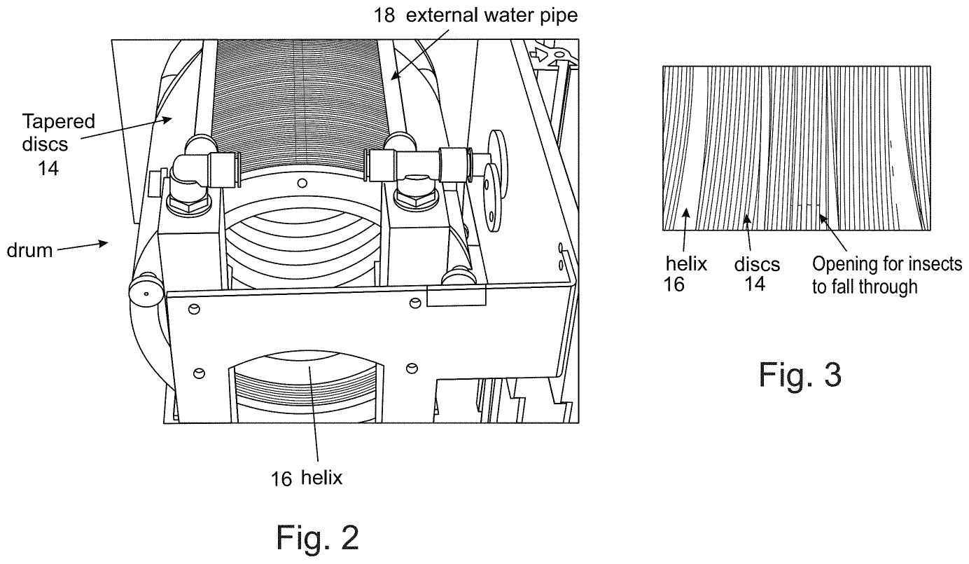

Reference is now made to FIGS. 1 and 2 which are side perspective and upper front perspective views respectively of a sorting drum 10 according to the present embodiments, the controller and counting device not being shown.

In the embodiment of FIG. 1, Flow of insects enter the drum 10 from a first side 12 on the right side of FIG. 1, denoted “Entrance point for pouring unsorted pupae”.

Females propagate along the drum 10, and exit at a female exit point (not shown), while males are flushed downwards. The embodiment shows two different areas to flush out pupae depending on their body size. Males are collected beneath and along the drum, and females are flushed out at the other side of the drum.

The wall of the drum comprises tapered discs 14, and those insects having a body size that can be pushed through the spacing between any two tapered discs 14, will fall out of the drum towards a collection area beneath the drum.

However, larger insects, and the female pupae are larger, cannot move through, and instead become stuck inside the spacing, eventually causing clogging to the opening. Clogging prevents the smaller insects, that is the male pupae, from being flushed out through the spacings. The males get stuck above and between the female pupa and do not fall through, causing a reduction in the efficiency of the device. As the process continues, this can lead to the loss of many male pupa that fail to get through the tapered discs and instead continue to flow along the internal side of the drum, along the tapered discs together with the females until they are flushed out together with them.

A second undesired phenomenon, is due to having a flow of liquid together with insects, flowing above small spacings from one side of the drum to the other. The flowing liquid may flow too fast above the spacings, and many of the insects may flow due to inertia together with the liquid towards the second end, and simply be washed out together with the females without getting a chance to fall through the spacings. However, if the flow of water is too slow, all the water may then be flushed down in between the spacings due to gravity, and no water reaches the end, so that the spacings fairly quickly become clogged with both females and males above the spacings without additional water to push them onwards.

The use of a helix 16 as a screw conveyor as an internal part of the drum ensures a continuous slow and controlled propagation process of the liquid together with the insects towards the exit. In addition, rotation of the drum solves the clogging issue, and finally the continuous flow of incoming water along the internal side of the drum and above solves the problem of water being flushed down through the openings with no new water to propagate along the drum.

As the drum rotates, an external water inlet 18 may pour water along and above the drum tapered discs, flushing the clogged up larger insects (e.g. females) back into the drum, while the helix geometry inside the drum supports propagation of both the females that are now flushed back into the drum and the material (males and females) that did not get through the spacing, to slowly propagate along to the next spacings, providing additional opportunities to sort the males before the remaining ones that were not flushed out may exit together with the females at the far end of the drum.

Reference is now made to FIG. 3 which is an insert showing the wall of the drum made up of spacings between the tapered discs 14. Parts of the helix 16 may be seen passing in between:

The spacing in between the discs above the male collection area is constant in this example. They are formed by placing aluminum spacers (other materials are possible as well). The spacings may also vary from a minimum allowable spacing for the males, the spacing that ensures that males only with that minimum size will go through the opening, to the maximum allowable spacing for males, enabling a larger population of males to be sorted, but at the same time increasing the chances for higher levels of female contamination within the male collection area, e.g. male container.

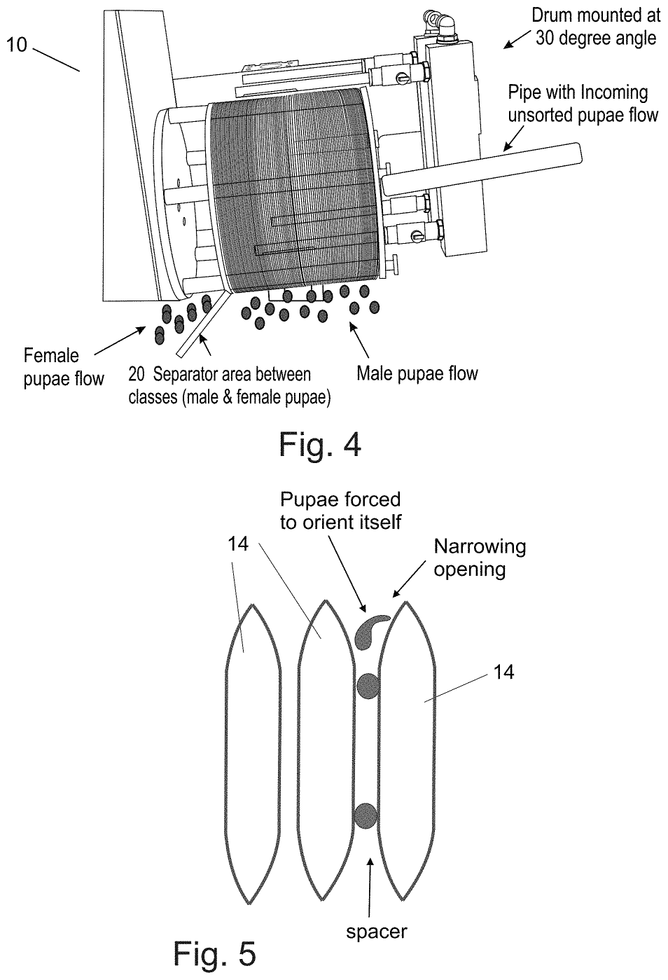

Reference is now made to FIG. 4, which illustrates a flow of male and female pupae coming out of the sorter drum device 10. A separator 20 may be set up between the male and female exit areas to enable better collection of each class of insects. Such a separator may be for example a plate or a funnel that supports the movement of one class to a different position, or the movement of each class to different collection area. The drum as described above may be mounted at an angle as shown to support the flow of material.

Reference is now made to FIG. 5 which is a view of a longitudinal cross section of discs 14 mounted next to each other to form the wall of drum 10. The discs have an initially convex surface which then flattens towards the middle so that when two discs are positioned adjacent to each other they form an opening which narrows from a relatively large initial opening and then widens again at the bottom. An advantage of such a shape is to move the pupae into a body orientation that can more easily be pushed through the opening and flushed down between the discs.

FIG. 5 shows the outline shape of a single disc 14 and shows how the shape influences the spacing between them, forcing the pupa to orient itself into a position in which it can be pushed through.

The series of discs represent a series of openings one adjacent to the other through which the material to be extracted may pass through, while the remaining unsorted material on top of the openings may continue to be conveyed along the opening until it reaches the position in which the second type of material from the unsorted material (e.g. female pupae) can be collected.

The openings may be adjacent and continuous without direct connection between any two openings, or separate openings may be connected to each other using a connector, for example a spacer.

It is now explained in greater how clogging may occur, and how it may be dealt with.

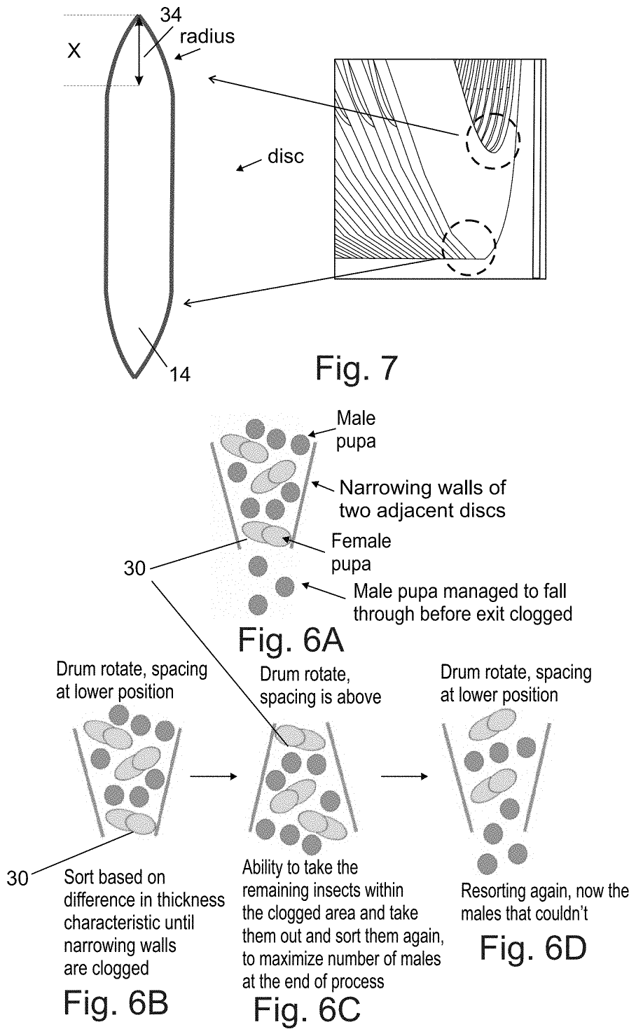

When the materials water, male pupae and female pupa, and possibly also male and female larvae, enter the tapered discs, male pupa initially manage to fall down through the opening, but after some time, female pupae may clog the opening, preventing other male pupae from being flushed out through the opening. FIGS. 6A-6D show how the clogging is resolved by rotating the spacing so that the narrow spacing is below the wide entrance in A, B and D and above in C and female pupa 30 that has got jammed in the space in A and B is dislodged in C, allowing free fall of male pupae again in D. Along with inverting, the gap is flushed with water, enabling male pupa to enter the next spacing and increasing the chances of their being flushed out. The process repeats itself until the males are flushed out, either through the designated area for males, or they are flushed together with females at the sorted female area.

As the water continues falling due to gravity in between the spacings, additional water enters continuously to flow inside and along the drum through internal water inlet 32 (FIG. 1). The internal water inlet pipe ensures that water gets to all of the spaces rather than pouring down the first space encountered, and ensures that there is water left to propagate the material horizontally.

As long as there is sufficient water with pupa the helix 16, or helical screw, propagates the material slowly towards the second end. As the material propagates, pupae are pushed in between the next tapered discs, while pupae that did not manage to be pushed inside continue to propagate.

Reference is now made to FIG. 7, which shows a modification for minimizing the clogging phenomenon by reducing the X dimension of the concave part of the disc, indicated by arrow 34, enabling only a few pupae to go through, and having the advantage that when clogged by a female, only a few will other pupae are caught behind the blockage since there is less space. In a prototype example the distance denoted by arrow 34 was set at 20 mm.

Additionally or alternatively, the drum may be vibrated to clear the openings and deal with clogging.

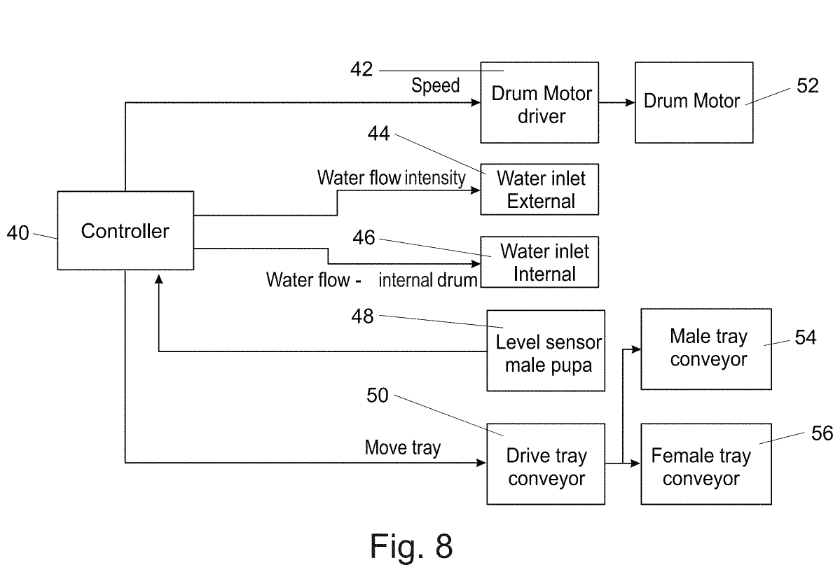

Reference is now made to FIG. 8, which is a simplified block diagram illustrating a system controller for controlling the process of separation of the pupae into male and female and then placing the males into different male pupae containers.

The controller 40, for example a computer or dedicated chip or ASIC running a computer program, or a dedicated PLC controller with a program built in, commands the drum to rotate at a specific speed. The controller ensures empty containers are ready under the male and female sections to be filled as the insects arrive and are changed when full. The controller may obtain readings from a counting device, to get an idea of how full the container is, or may receive a trigger signal, say associated with lapsed time for filing a single container, or a number of pupae in the container may be sensed to have reached a pre-defined value. Upon determining that the container is full, the controller commands the conveying system to move the specific container towards its next position and bringing an empty container underneath he drum in its place.

The controller 40 controls a drum motor driver 42 that rotates drum motor 52. An external water inlet valve 44 controls water flow for flushing. Opening and closing of the pipes that deliver internal water with pupae is controlled by valve 46.

As mentioned, once the controller receives information from the counting device or level sensor 48, the controller may decide that the male pupa tray (container) is filled with a sufficient number of pupae, and may command the container drive system 50 to move the male container forward, towards the male collection area. Movement of the containers may involve a conveying system, male tray conveyor 54 or female tray conveyor 56, which may be rail conveyors or a simple conveyor on top of which the containers are either fixed to the rails and propagate by having the rails move, or they are positioned on the conveyors and are conveyed (propagated) due to the movement of the conveyor. The skilled person will be aware of numerous ways in which to propagate items as part of a production line.

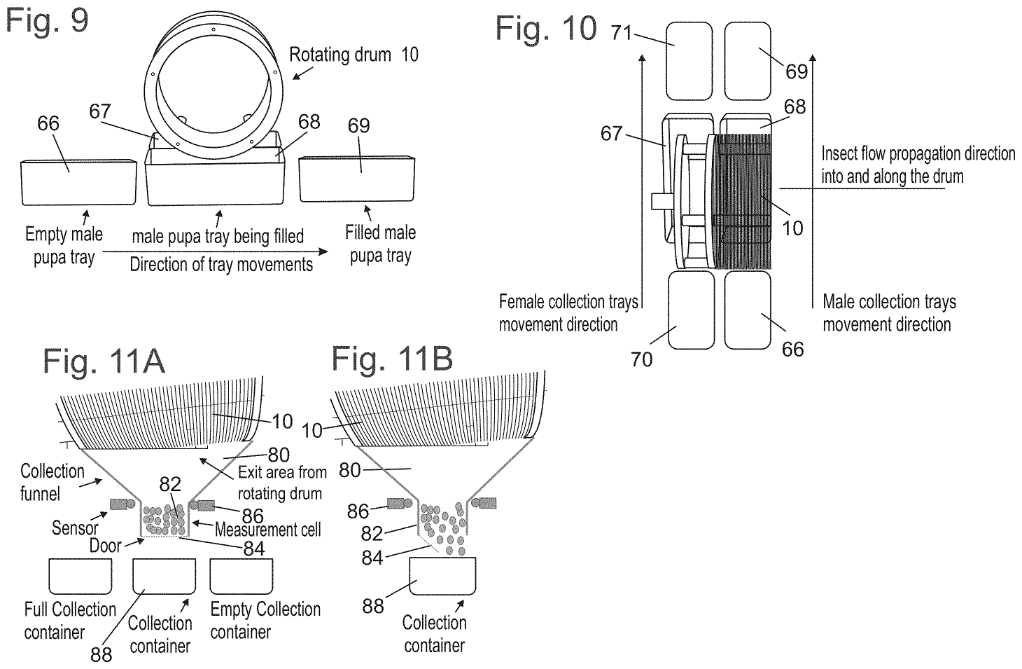

Reference is now made to FIG. 9, which illustrates the placing and movement of containers 66-69 around the drum 10. The controller carries out synchronization involving filling the male pupae with sorted male pupae falling from the drum in container 68. Container 67 at the rear opening collects the females. Then, when the container is filled, the containers propagate to the right 69, with a new empty container 66 being positioned under the drum 10 to collect the next batch of male pupae. The controller may in one embodiment decide the container is filled based on readings coming from a level sensor.

FIG. 10 is a top view that shows the simultaneous movement of the containers per each different class, male containers 66, 68, 69 and female containers 67, 70, 71 in this example. The movement of both queues may happen at different speeds and not at the same time, since the filling rate at the female container and male container may be different. Also the containers may move in different directions as they go to different destinations.

The present embodiment may enable sorting into at least two different classes and filling two different collection areas at the same time, hence even further extending its benefits. Each queue of collection trays may move in sync with the rate at which at which the current tray is being filled under the drum.

Reference is now made to FIGS. 11A and 11B, which depict an option for any one of the present embodiments to count sorted male pupae that exit the drum, so as to allow the controller to know who full the current container is.

The underside of drum 10 leads to a connection funnel 80 at the bottom of which is a measurement cell 82. Pupae released from the drum pile up in the measurement cell while door 84 is closed. As the pupae reach the level of sensor 86 the door is opened (FIG. 11B) and the pupae are released into current collection container 88. Both ends of the measurement cell 82 enable the flow of water through them, while in the closed position of the second end, they prevent flow of pupa through the second end. The door 84 may be a porous plate with small holes through which the male pupae cannot go through, for example smaller than 0.5 mm. It is noted that the smallest mosquito male pupea, those of Aedes albopictus, have a typical size of 0.89 mm for the cephalothorax, the thickest body part.

Sensor 86, located next to or at the neck of the measurement cell 82 senses the level of pupae grouped inside the measurement cell 82.

The sensor for example may comprise an LED on one side, and a light detector on the opposite side. When the light detector does not detect the led light for more for a pre-determined time (e.g. one second), it is implied that pupae are blocking the light, and hence have reached the desired level.

The process of filling an empty container with a new batch of counted pupa is as follows:

The controller sends a command to a measurement door to be opened.

The controller sends a command to rotate the drum at speed A (e.g. 1 cycle per minute).

The controller sends a command to open the external water inlet and then the internal water inlet.

The controller sends a command to close the measurement door.

The controller sends a command to start pouring insects into the drum. At this point the insects are falling through the discs and towards the measurement cell and are slowly building up in the cell.

At some point, the sensor 82 stops seeing light across the neck of the cell for a certain amount of time (e.g. 1 second) and at this point the measurement cell door is opened as a particular number of pupae has now been reached.

Thus now the pupae from the measurement cell are flushed down towards the empty container.

It may take one or more cycles of opening the door to fill a container. Once the specified number of openings has occurred, then after say 3 seconds the door is closed, and the now full container is moved towards its next position. A new container is now conveyed towards the filling position located underneath the measurement cell.

In FIGS. 11A and 11B there is only a single element that is used as a door with axial movement at the lower end of the cell. Adding another door to the neck of the cell, or the entrance area is possible and may also result in a more accurate count, since once the counting of the pupa inside the measurement cell reached a certain value, the first end can then be closed as well, preventing additional pupa from arriving, and keeping an exact number within the measurement cell.

It is possible that once the controller has determined that the measurement cell is full, and has flushed down its content, an identification may be sent to an operator, who may manually take away the now filled container, and replace it with an empty one ready for the next filling.

It is further possible to have the container being filled positioned directly below the rotating drum without having the funnel and/or the measurement cell and sensor. In such a case, propagation of the containers may be performed automatically based on a timer, or other trigger, or even manually.

The collection container (referred to also as a tray):

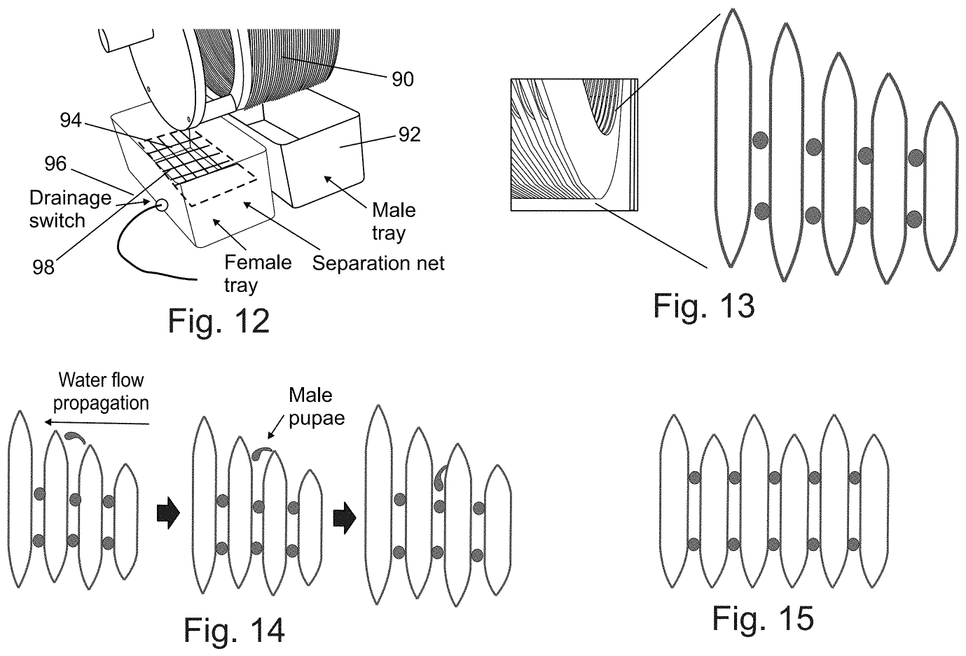

Reference is now made to FIG. 12 which illustrates part 90 of a drum with male 92 and female 94 collection containers in position underneath. As the water fills a specific collection tray (either a male tray or female tray), and to prevent a situation in which the water level exceeds the height of the tray and the tray overflows, a mesh structure 96 (e.g. a net with grid cells smaller than 0.5 mm to ensure only water goes through and not pupae of any sex) may be placed at some height above the floor surface, and a switch (valve) 98 may be provided to enable drainage of the water. Water may get through the mesh, while the pupae do not, and the water is then removed. A pump (not shown) may be used to reuse the overspill and the water may be provided to the external water pipe for re-use as a source of water to be poured above the drum discs through the opening. In FIG. 12 the net is drawn only on the female tray, but would typically be placed in both trays.

Thus there is provided an embodiment in which a rotating drum contains a series of narrow openings with a radial orientation through which mosquito pupae of a first smaller size are flushed through by water, whereas mosquito pupae larger than a specific size cannot be flushed through with water because of their size. A conveying element such as a helix or helical screw may convey the pupae across the drum and rotate the openings to a second orientation at which any pupae that clog the openings are flushed away by the introduction of water coming inside through the openings. The pupae that are small enough are flushed towards a series of narrow openings formed by the discs in a sorting process. The sorting process may be repeated by feeding once sorted pupae a second time through the drum.

The sorted pupae are collected and conveyed, typically in a direction parallel to the axis of the drum. The drum rotates from bottom to top to remove the larger pupae that clog the openings, where water from above the discs pushes them back into the drum for repetitive sorting cycles. Conveying may happen in a perpendicular axis as well as will be shown later, as part of other potential variations.

A continuous pupae sorting device thus comprises a series of rotational openings positioned above a collection tray. The rotational openings enable repetitive and continuous sorting processes by pouring water down the opening from the opposite opening above.

The method comprises sieving unsorted material (pupae) through a narrowing element; driving (convey) the element toward a flipping (rotation) position; flipping (rotate) the element; empty the element; flip the element back to the initial position; drive (convey) to the next position for refilling and repeat the process.

When moving the element described above, instead of flipping, then after first sorting, the element may be conveyed towards a second location, and there the element may flush clogged material, after which the element is moved to be filled again for iterative sorting. It is possible that instead of moving the element, the collection tray located below may be moved in sync with the status (orientation) of the element. Thus for example when a wide area is on top a female tray is located below, and when the wide area is oriented below then the male tray is located below.

An apparatus according to the present embodiments may sort material with at least two different material classes to be sorted, the apparatus having a series of openings one after the other in first axis, the openings having an open area in a first and second axis, the open area being wide enough to allow material of the first class to pass through in a third axis perpendicular to the first and second axes and prevent the second class of material from passing through the opening in the direction of the third axis. A conveying device may convey unsorted material along the series of openings from the first end of the device to the second end.

The material may be made up of any combination of female mosquito larvae, male mosquito larvae, female mosquito pupae, and male mosquito pupae.

A conveying device can be for example a motorized screw conveyor in the shape of a helix.

The openings may be narrowing openings, with exemplary size 1 mm, to allow typical male pupae to go through while preventing typical female pupae mosquito from going through.

The set of openings are comprised by having a series of discs spaced between each other. The separations may be defined using spacers.

Collection containers may be provided as well as a conveying system to convey the containers to locations below the openings to collect the falling insects. Conveying the containers may be carried out in sync with a sensor that helps to indicated when the falling insects have filled the current container.

The sensor may be an imaging camera capturing an image and detecting a number of objects in the image and accumulating the number of objects that pass between two points in time. Additionally or alternatively, the sensor may be a light sensitive sensor opposite an LED and counting is of events in which light is blocked due to falling insects blocking the line of sight. Given the pupae length, the height of the fall and gravity an estimated amount of time for a single pupa to traverse the sensor FOV can be calculated, from that a total time of blockage may be translated into a total number of pupae.

The device may include a controller unit to automatically control the drive components in accordance with the different feedback signals received from the sensors in the system.

The device may have a series of openings and a screw conveyor and a mechanism to wash out clogged insects inside the openings.

As discussed above, a mechanism for washing out clogged insects may be provided by making the drum rotate. The series of opening are the edges of the rotational drum, and such a mechanism is a vertical up and down motorized conveying device that raises and lowers the openings as they are held together in a frame, into a water container and may allow water to push through any clogged openings.

The drum may be mounted at an angle to support the flow of liquid and unsorted pupae between openings towards the drum outlet.

The device described above can be operated manually. In this case a person may manually rotate the drum, and manually control the opening of the valves to let water bearing the insects get inside the drum. The person may likewise open valves to let water pour inside the drum, say via the inlet water pipe and externally above the drum. The person may also manually change the collection trays when full.

The drum may in such a case consist of the same elements as described above, only without automatic control of the drum rotation or counting. The level of automation may thus vary from complete manual to semi manual to fully automatic. In the semi-manual case a sensing element may sense the level sensor and upon reaching the pre-defined level may notify the user to change trays, while rotation of the drum can be automatic. The fully automatic case is the embodiment described in detail with reference to FIGS. 1-12 above.

Reference is now made to FIGS. 13, 14 and 15 which illustrate how, in order to support slow progress of the material (the unsorted pupae) between discs, the drum may be constructed of a series of discs with different sizes, such that their dimensions increase along the drum. Furthermore, such a structure of ever increasing height of the discs edges along the propagation direction of the insects, supports the chance for insects to be forced to orient their bodies towards the opening below, rather than flowing with the liquid simply along drum.

FIG. 13 shows a sequence of discs 14 whose height increases into the depth of the drum.

FIG. 14 shows a sequence of three stages in which a pupa approaches an edge, and then because of the water flow, gravity and the radius of the disc edge is forced to orient itself downwards so that in the third stage it is oriented so that it is able to move in between the spacings.

FIG. 15 illustrates a sequence of discs in which repeated sizing sequences are used.

As discussed above, the main building blocks of the drum include spacings with openings that narrow to allow pupae below a certain size to move through whereas other larger pupae may not pass. A conveying mechanism conveys the insects along the openings, the insects then either fall through the openings to be collected below, or continue along the openings until they exit at the area corresponding to larger size insects.

The spacings in the above discussed embodiments are created by having discs attached to one another using spacers with either constant or changing size and hence the same or different sized openings. However, other ways are possible to create spacings with narrowing openings, such as using a 2D plate with drilled holes etc.

Then a variation of the device in keeping with other features mentioned above for sorting of the male pupae is a device made up of a frame to hold a porous surface. The surface is comprised of grid cells. The cells have a narrowing opening. The openings are of constant dimensions from side to side. A screw conveyor such as a motorized helix is mounted above the surface, and unsorted pupae with water are poured at one end, and as the helix rotates, the small insects fall through the openings, while other insects propagate towards later openings.

As water continues to pour on the surface, the larger insects which may have got stuck at the entrance to the opening, are flushed away towards a larger pupae area for collection.

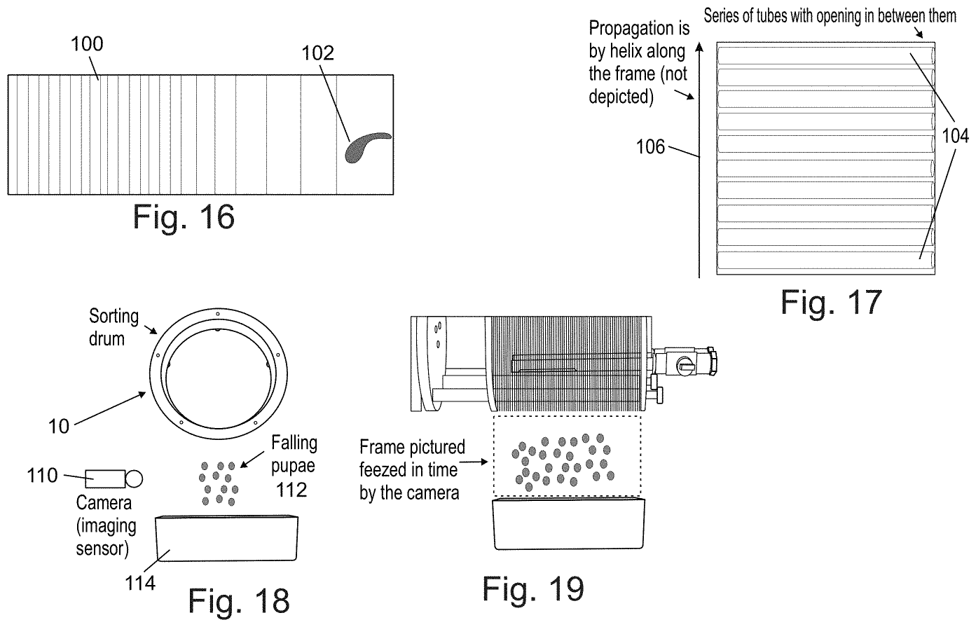

Another option is having a grid with changing size of grid cells along the flow direction, as depicted in FIG. 16.

In another variant, in order to ensure minimum potential clogging, the porous surface is positioned as part of a frame holding it and may move up and down, say by means of a motor moving the surface up and down on a rail conveying element. Thus, while the helix rotates, the entire surface may move slowly up and down. In the down position the surface may be below water level so as to fill the container, and while in its upper position it is above the water level. Small pupae are flushed down through the narrow openings into the container below, and as the surface moves down into the water, water rises up through the openings pushing up any potential larger insect that may have clogged the opening for removal by the rotating helix. FIG. 16 depicts a top view of a frame 100 with a series of openings with varying dimensions, and a large pupa 102 falls through the last opening.

A further variation of a porous surface is a 2D grid, made of mesh with small openings through which the small insects can fall through but the larger ones cannot. The principle is similar to the above, including having a helix or other screw like conveyor element to propagate the material along the net, but the cells in this variation do not have narrowing openings but rather fixed size openings.

In another embodiment, unsorted pupae are poured from above onto a mesh, and the mesh may sieve the pupae through. Then, after a pre-defined time or an amount of unsorted pupae poured on the mesh surface, pouring stops, the mesh is moved to the side, flipped, say by turning over the openings, and water are poured from above. Clogging is removed and the mesh is returned to its position and the sorting procedure repeats. Below the mesh is located a collection area for collecting the pupae that have fallen through the mesh.

A device for pupa sorting may have rotational openings around an axis parallel or perpendicular to the lengthwise dimension of the set of openings, as well as an ability to be raised or lowered along another axis, say perpendicular to the lengthwise axis of the set of openings. Raising and lowering may support emptying anything clogging the openings, and switching may be carried out of collection containers below the set of openings in sync with the status of the rotational openings. Thus, when only small pupae can get through, a first type of collection tray is below, and when the openings are rotated and thus only larger ones can be flushed through, then before they are rotate, the collection tray is switched with a second collection tray, and after rotating the openings again, the collections trays are switched again. Switching the trays may be manual or automatic as may other aspects of the operation.

Collection trays may be alternated below the openings. In one embodiment the openings are rotated but are still above the same position and male and female collection trays are moved each time to the left or to the right. The trays may be held on a simple conveying element (e.g. motorized rails). In another embodiment the male and female collection trays are static, and the openings may be moved above and between the different collection trays matching the state of the opening orientation.

Conveying of the pupae may be in along an axis perpendicular to the axis through which the pupa are pushed, for example in one case described herein a series of tubes distanced from each other extend over the length of the collection tray in position. The series of tubes may be connected in a chain like configuration, forming a loop, and when they reach the upper position, water pours down to push the clogged pupae for further sorting.

Another option is that the connected tubes do not form a closed loop, and the propagation is implemented by a helical screw located above the tubes to propagate the pupae for iterative sorting through multiple openings. At the end, pupae that are too large may fall out at the position designated for the larger class.

Continuous flow of water from the input end of the device pushes out larger pupae that cannot pass through the side openings to the far end of the device.

As shown in FIG. 17, the tubes 104 may be separated from each other across the direction of flow 106, and not necessarily connected. This is achieved by having a frame, the series of tubes being connected to the surrounding frames.

In an example the elements forming the spacings along the frame are mere ropes, e.g. sewing ropes, or very thin nylon ropes, and the repeated sorting of the pupae is then achieved by the screw conveyor (e.g. horizontal helix) located above.

Reference is now made to FIGS. 18 and 19, which illustrate another option for counting the falling pupa, particularly useful for the rotating drum embodiment. An imaging sensor 110 (e.g. a camera) is set up with a FOV over the falling pupae along the entire area beneath the drum 10 from which the pupae 112 fall. Alternatively, multiple cameras may cover the entire underside of the drum with a composite field of view. The camera may capture the falling pupae and use object detection to determine the number of falling objects in each frame. With high frame rate, that is multiple frames per second, it is possible to capture many if not all of the falling objects. Accuracy to the level of a single pupae is not necessary.

Once the algorithm detects a pre-defined number of falling objects towards the collection area, then the controller may send a command to replace the now filled collection tray 114 with a new empty collection tray. The controller may also send a command to stop the flow of insects into the rotating drum while the trays are exchanged. The controller may also stop the flow of water, whether external or internal. FIG. 19 is a conceptual view showing how the camera may capture falling pupae in a frame.

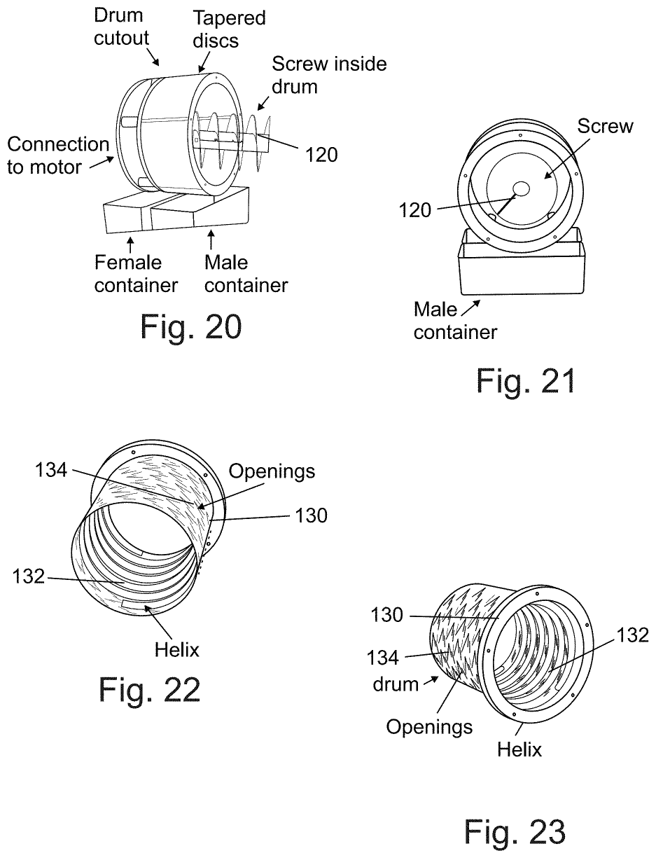

Reference is now made to FIGS. 20 and 21, which are side perspective and end-on views respectively of a further embodiment of the present invention. In the embodiment of FIGS. 20 and 21, instead of having the helical screw extended into the drum and reaching the internal walls of the drum, that is reaching the discs, in order to move the water with unsorted pupae through the drum, helical screw 120, which may be with or without a shaft, may be integrated into the drum. As before, both drum and screw may rotate. The screw rotates in order to propagate the material, while the drum rotates to enable emptying the clogged spacings.

That is to say, in some embodiments, the helix parts may be installed between gaps in the slots or just inside thereof. They may be welded or simply held by pressure in between the discs.

Another embodiment may entail building a helix separately, and then pushing it into the drum, so the helix edges are just above the disc edges. The structure is then inserted into the drum to build an integrated structure.

In the integrated structure, the disc diameter is larger than that of the helix. The discs are installed first and then the helix is pushed inside and then a closure is fixed on which holds both the discs and the helix in place.

A further embodiment uses a propeller located at the entrance of the drum. The propeller pushes the water and pupae, because of the force applied by the rotating blades of the propeller, forwards towards the second exit, and while the material is being propagated, it falls down through the openings to be sorted.

Reference is now made to FIGS. 22 and 23, which shows a further variation of the present invention. In FIGS. 22 and 23 the drum structure has a wall 130 that integrates the helix for driving purposes with openings for selecting the pupae. A helix 132 is built into the inside of wall 130 and openings 134 are built into the wall 130. As the drum rotates, the helix 132 drives the material forward along the drum.

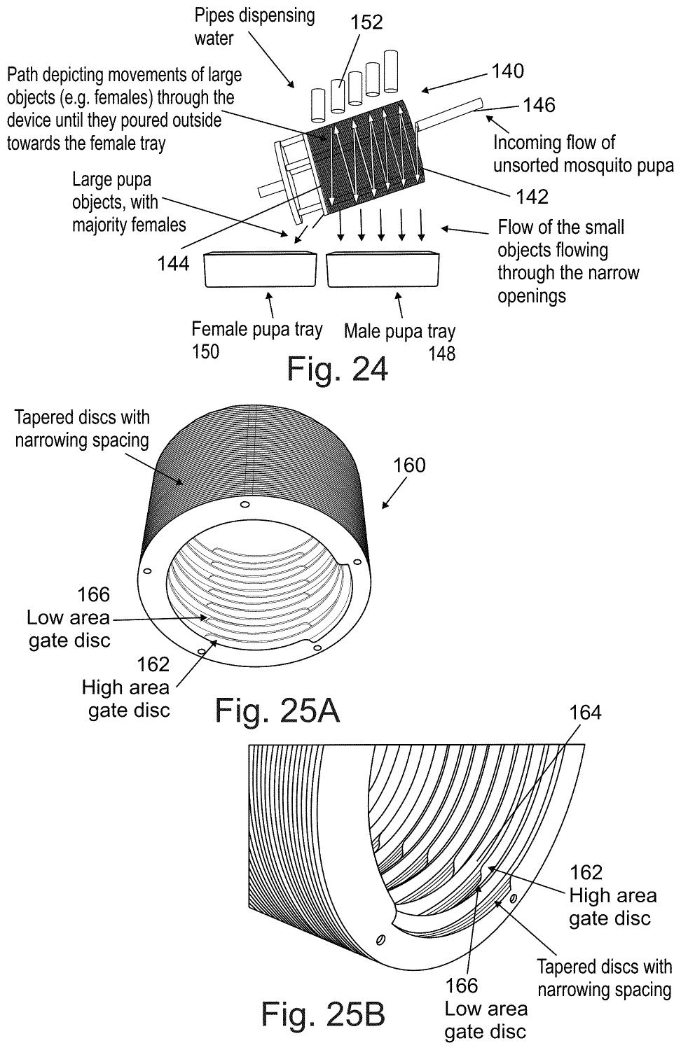

Reference is now made to FIG. 24, which is a simplified diagram showing a further embodiment of the rotating drum. The rotating drum device may be provided without an internal helix. As in previous embodiments, the drum 140 is mounted at an angle to support flow of the water and insects. Insects mixed in water enter through the raised end 142 of the drum and move towards the lowered end 144, with the males being filtered out along the length of the drum into male collection tray 148. Females reach the end of the drum 144 and are collected in female collection tray 150.

In one embodiment, the inlet of incoming pupae pipe 146 is in the plane of the drum entrance at end 142. However, it is possible to have the pipe extend within the drum and dispense pupae along the entire length of the drum. Pipes 152 dispense water into the drum to help with flushing the pupae through the drum and to prevent clogging.

Reference is now made to FIGS. 25A and 25B which show an embodiment in which the sorting device is made of a rotating drum 160 consisting of discs separated from each other to form the openings as before. Some of the discs extend further into the interior of the drum than others. Such discs, denoted gate discs 162, serve as separators to form circumferential channels 164 around the interior of the drum. Other discs, 166 are of lower height and form the interior of the channels, so that each channel has more than one opening. Thus in effect water below the level of the disc height at that area is prevented from passing in the axial direction of the drum, thus forcing more pupa to try to be pushed in between the openings instead of simply flowing from one side to the other side of the drum. As the discs continue rotating, an area with lower height is now positioned at the lower area of the drum, and enables the flow of water and unsorted material in the forward direction within the channel formed by the gate discs. The variable height discs may be used in conjunction with the helix of any of the other embodiments herein.

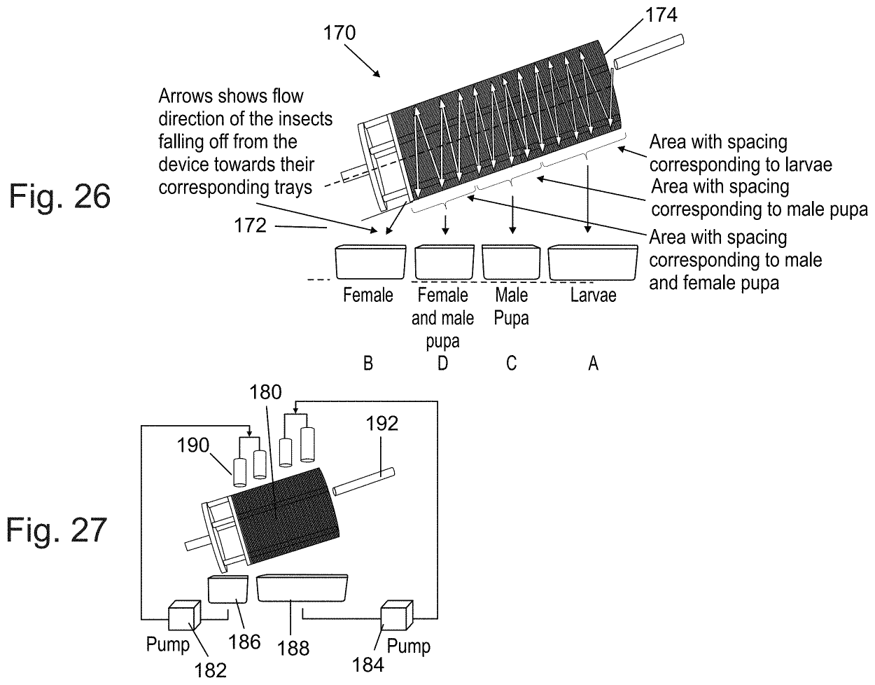

Reference is now made to FIG. 26, which shows a variation 170 of the drum which is divided into two or more sections, each section having different sized spacings to filter a different size of insect. For example there may be one section A for larvae, one section C for male pupae, one section D for an intermediate size where the pupae may be small females and large males together and the opening at the end B for females.

A female collecting tray 172 may be then located under the female section corresponding to wider spacings, and a position for exiting the drum may simply be the end of the drum.

Alternatively, a large spacing may be provided between the two end discs above the female tray, so that they too have their filter opening. The drum 170 thus only requires one opening on one side for the entrance of the non-sorted insect. The drum may nevertheless have a second opening at the lower end, and then females may fall through both the opening between the discs corresponding to the female section, and from the end, thus avoiding clogging issues with the female section.

In FIG. 26, the drum is actually divided into three sections, with the females exiting from the opening at the lower end. Pipes for water flow are not shown, although pipe 174 for dispensing of the water-pupa mix is shown. Objects, typically the mosquito pupae, that have a body size larger than the largest spacing in the first collection area, pass on to the second collection area, and so on to the end of the drum.

Reference is now made to FIG. 27, which is a simplified embodiment of a variation 180 of the drum, in which pumps 182, 184 are connected to one or more of the collection trays 186, 188, to move the water upwards to external water heads 190 so as reuse water to flush out rotated clogged insects in the drum. Pipe 192 provides the insect-water mix for sorting in the drum.

The angle of the rotating drum may be altered to match with the type of mosquito species being sorted.

As discussed with previous embodiments, rotation of the drum may be manual with a person rotating the drum and may also be motorized by attaching a motor to automatically rotate the drum. The rotation speed may be modified to achieve a balance between incoming flow, drum angle and rotation speed to optimize the sorting process. Thus it may be determined that a certain rotation speed gives a maximum number of sorted pupa of type A (for example males) falling through the discs, while having a minimum number of pupa of type A exiting from the end of the drum with a maximum number of pupa of type B (e.g. females).

Drums may be of variable length. Individual sections for different insect sizes may be added together as required. Within each section, spacings may be fixed, or there may be a gradual spacing increase from a minimum value to a maximum value.

The use of multiple sections or gradual changes in opening size provides the ability to have a region dedicated to the smallest possible males, increasing the probability for zero females. Later sections may correspond to larger males, but will have higher probability for including small females.

It is noted that in Sterile Insect Technique projects it is preferred to release competitive males, who will be the larger males, since size corresponds both with longevity and competitiveness in mosquitoes.

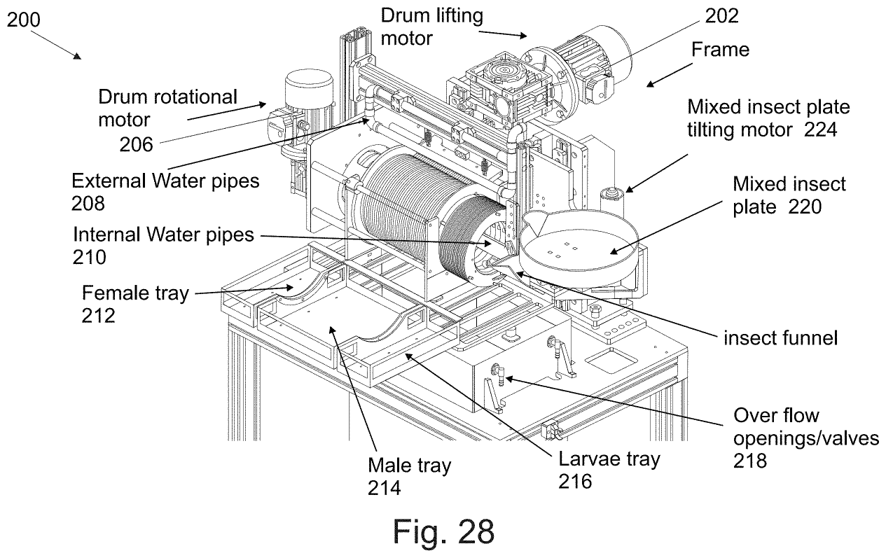

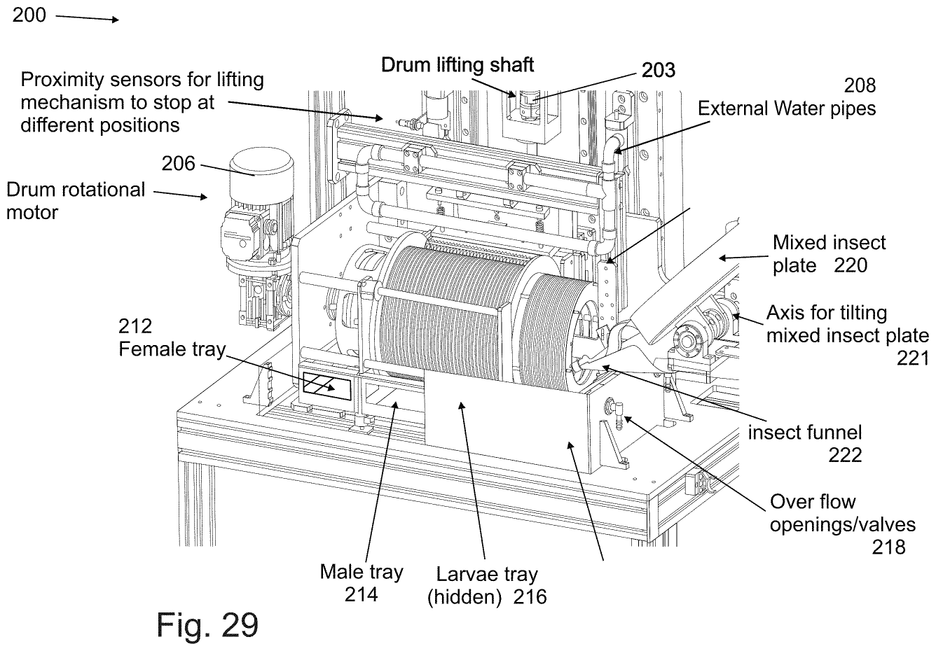

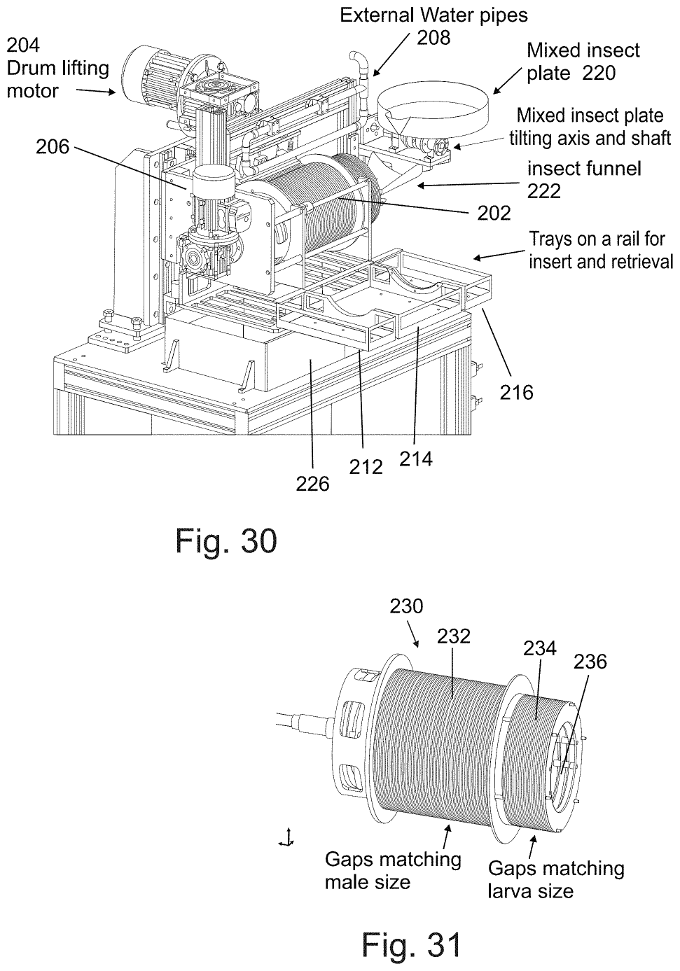

Reference is now made to FIGS. 28, 29 and 30, which are three different views of an integrated machine including a drum according to present embodiments. Machine 200 includes drum 202 held by drum lifting motor 204 via drum lifting shaft 203. Drum rotational motor 206 rotates the drum 202. External water pipes 208 provide water flow for flushing the openings in the drum. Internal water pipes 210 provide water for the internal flow in the drum. Female collecting containers 212, male collecting containers 214 and a larva container 216 slide into position under the drum. Overflow valves 218 allow water to flow out of the collecting area. A mixed insect plate 220 is fed with the mixed insects and water for sorting. The mixed insect plate may be tilted around an axis using mechanism 221. The mixed insects and water drain through funnel 222 to be fed into the drum. Frame 224 holds the construction together.

The collection containers are shown outside of the machine, and platform 226 under the containers may contain a pump, which may be connected by a closed loop to the external pipes 208 above the drum.

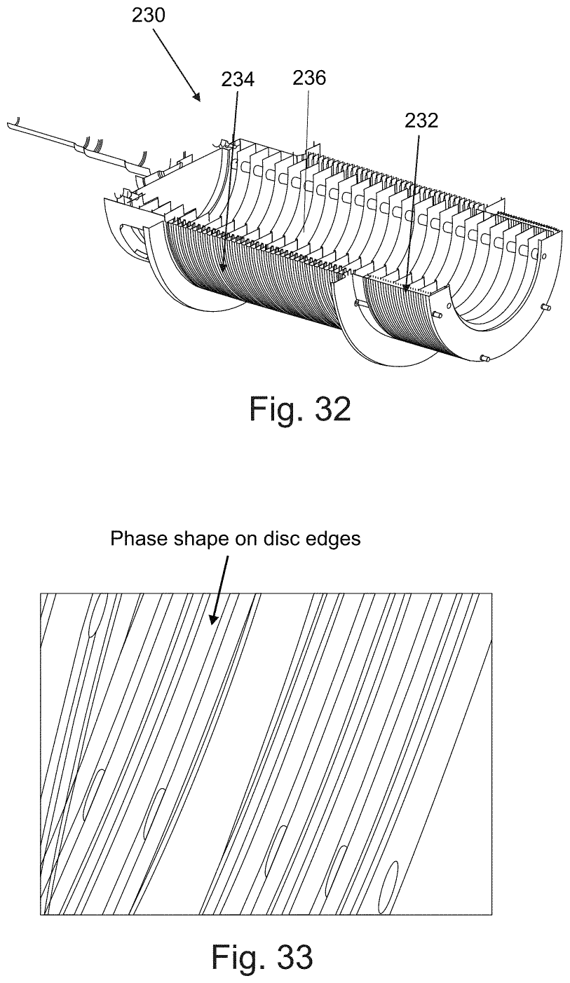

FIGS. 31 and 32 show a drum 230 according to the present embodiments having two sections, a first section 232 having openings for larvae and a second section 234 having larger openings for male insects. Helix 236 lines the inside of the drum. The helix extends along the structure from larva section towards the end, opposite the female collection container. The helix may end at the beginning of the female collection container or at the very end of the drum. FIG. 33 is an internal view looking outwardly towards the discs and showing the internal faces of the discs.

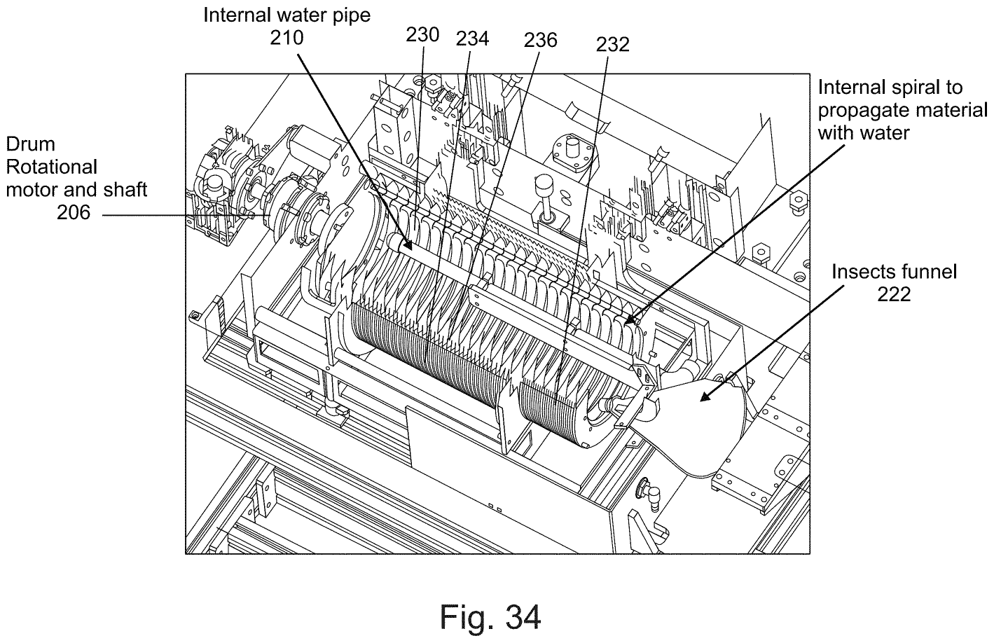

FIG. 34 is a cutaway view showing the drum 230 inside the integrated machine. The first and second sorting sections 232 and 234 are seen as well as helix 236. Internal water pipe 210 is also shown as are the drum rotations motor 206 and shaft.

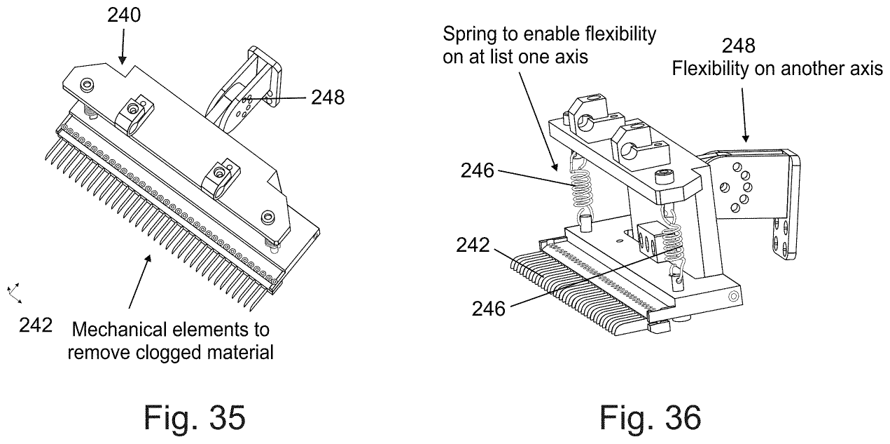

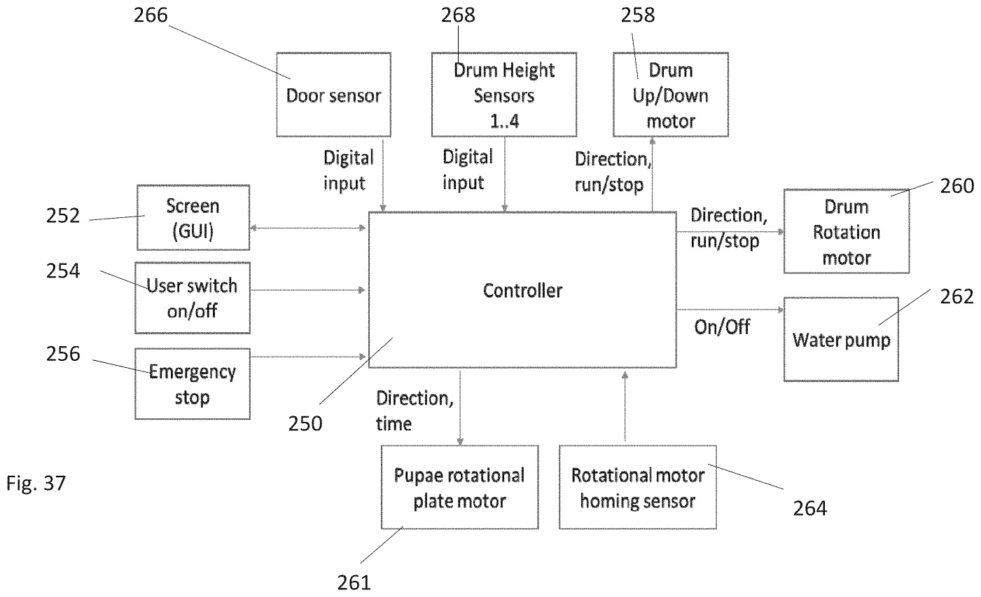

Reference is now made to FIGS. 35 and 36, which illustrate an unclogging element 240 having teeth 242 for insertion between the discs to clear clogged material. The element may be mounted in mounting device 244. A first pair of springs 246 provides flexibility on one axis. Flexibility on a second axis may be provided using flexible mounting bracket 248.

In use a technician places a batch of mixed insect material onto a motorized plate (the Mixed insect plate). The batch may include a mix of male and female pupae, or a mix of male and female pupae and larvae or any other combination.

The technician pushes into the machine a set of empty trays or collection containers

Reference is now made to FIG. 36, which shows the general architecture of a controller 250 for a machine according to the present embodiments.

The controller is interfaced using a GUI 252 and the user also has an on-off switch 254 and an emergency stop button 256. The controller can position the drum using drum up down motor 258 and rotate the drum using drum rotation motor 260 and the pupa plate 261. The controller may operate water pump 262. The controller receives sensing signals from rotational motor homing sensor 264, from door sensor 266 and drum height sensor 268.

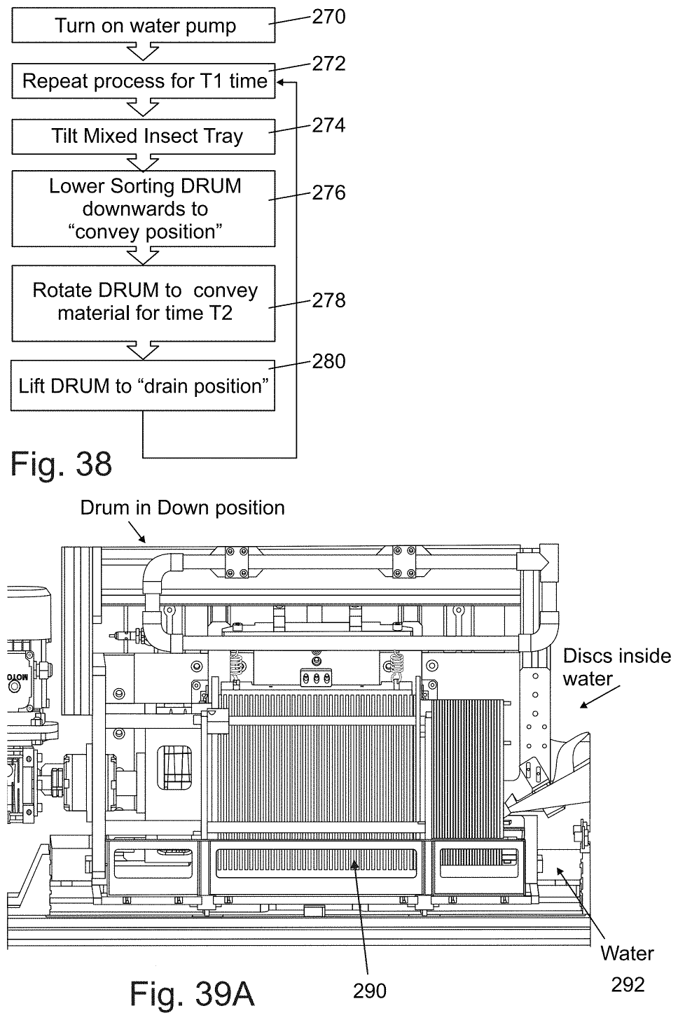

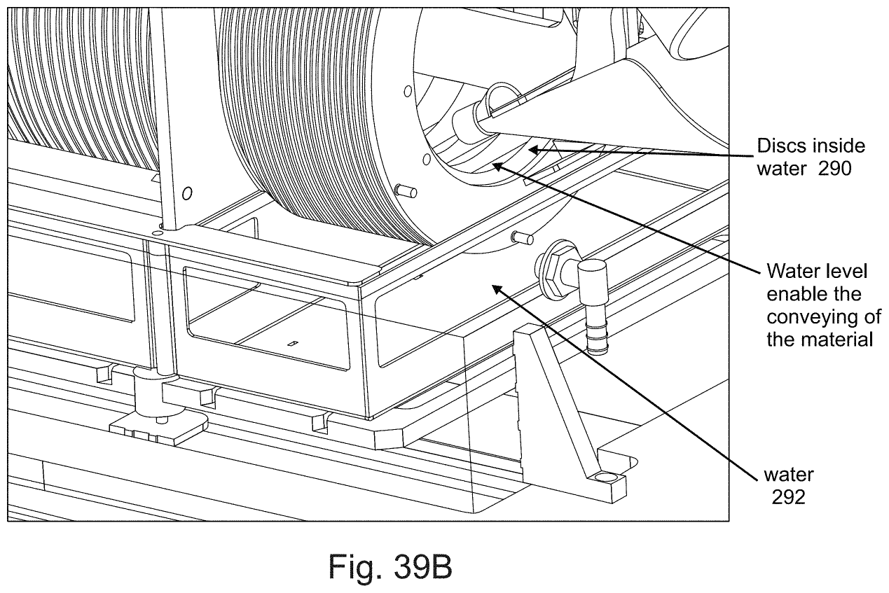

Reference is now made to FIG. 38, which is a simplified flow diagram showing a sequence of operation of a machine according to embodiments of the present invention. Upon start of operation, the following sequence is run as each element is triggered by the controller.

The water pump starts circulating water 270 and water is flushed along the internal pipe of the drum. Water may also run from above.

The functionality of the water from above is to support unclogging of females that have been jammed into the openings, and the water being flushed inside may flush the jammed insects downwards as shown herein. The process is repeated for a specified amount of time 272.

The mixed tray is tilted for a specified time 274. In embodiments, the tray tilting time may be measured in relation to position of the rotating motor, or another sensor may trigger the controller to stop tilting the tray. For example a camera may identify the amount of pupae already dispensed from the tray either above the tray or being directed towards the entrance of the drum.

Returning to FIG. 38, and in the conveying position, the rotational motor rotates the drum for a second pre-defined time T2-278. The spiral rotates as well, being connected to the shaft of the rotational motor.

The rotating spiral moves material along the drum until T2 ends at which point the drum stops rotating. Then the drum is lifted up 280 to the drain position, and the cycle repeats.

The controller continuously reads the position of the motor as it receives readings from the proximity sensors installed along the up/down movement axis of the drum. The drum is lifted just above the water level for draining, and as it is lifted, water is flushed between the discs.

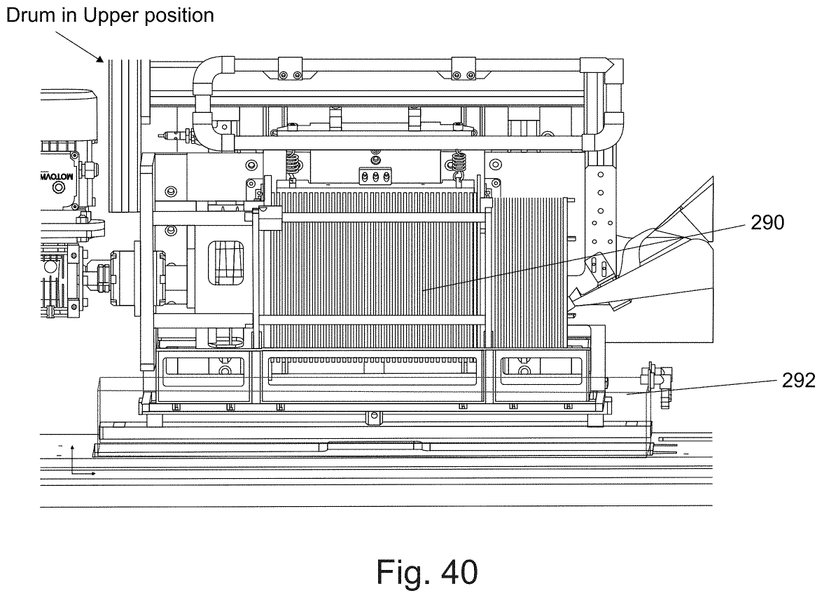

Reference is now made to FIG. 40 which is a view of the drum in the raised position. Discs 290 are above the water level 292. As the discs are flushed, insects whose size corresponds to the gaps between the discs are flushed out too. Helping the insects to fall down, is the combination of the shaping of the gap between the discs, and the continuous flow of the internal water. Some of the insects which are not well oriented may not be flushed out and may remain stuck along with the larger insects along the discs. Some may also get stuck in between the openings. The water being poured down as the drum is in the upper position also supports keeping the insects wet.

The process is repeated, again, for pre-defined time.

During the process, larvae are sorted and flushed towards the larvae trays at the first section which includes gaps between the discs smaller than 0.75 mm, since on average the male pupae width is about 0.8 mm-1.1 mm on average for aegypti. Other species may be of different sizes and may require different calibration. As the material continues, males are flushed in between the discs along the male section with gaps corresponding to 1 mm, at which size some small females may be flushed out as well, and then the rest, presumably mostly females, moves towards the female section.

While the females propagate along the male section, they may get stuck in between the discs, as mentioned. As the discs rotate, then on the upper area there is an unclogging mechanism to take out the females causing the clogging. The mechanism may comprise a high pressure water jet coming from the upper external water pipe. Alternatively or additionally, the mechanism may comprise mechanical knives spread above and along the male section, and connected to a fixture with a spring, allowing some degree of flexibility for the entire element to move up and down as it hits part of the structural bars holding the frame of the discs along their perimeter—see FIGS. 35 and 36 above.

If the clogging is not resolved then over time the entire drum machine becomes steadily less efficient.

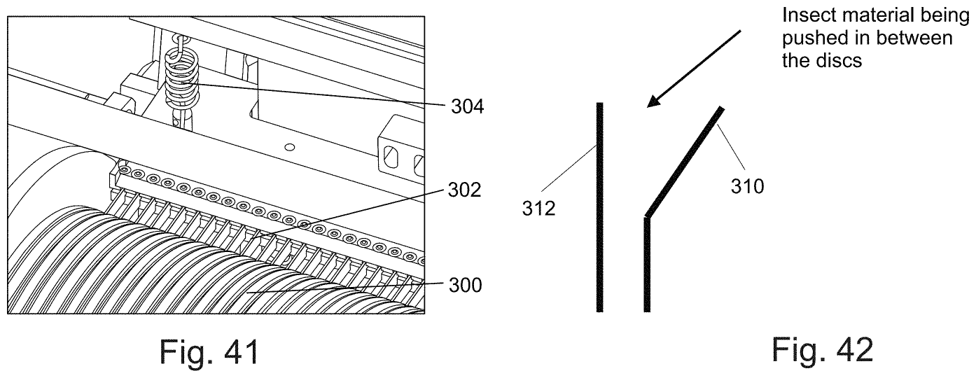

Reference is now made to FIG. 41, which shows the upper side of a drum 300 with a bank of knife elements 302 connected via a spring 304 to provide the arrangement with cleaning flexibility.

It is noted that the external upper pipe above the drum may also pour water while the drum is being lifted, so as to support pushing the insects downwards in between the gaps. Thus, in one embodiment, there will be only that an external pipeline along the drum carrying out flushing. In another embodiment, the internal pipe continues pouring water within the drum.

Once the time lapses, or a technician decides to stop, or some other trigger stops the process, say based on a weight detector, weight of insects in specific tray, a camera identifies an amount of insect material in the male pupae section or in other sections etc., then the drum is lifted to the upper, draining, position at which the now-filled trays can be retrieved and later switched with new empty ones.

The trays may include magnetic sensors on their backs in order to trigger a notification for the controller that the trays are in position/out of position. When the trays are out of position, the controller prevents the regular sorting process from running and may notify the user.

In embodiments, the dimensions may be varied. Thus, having a longer drum element may enable more potential cycles for the material to move in between the spiral cells prior to reaching the females area.

Instead of having the pupae being poured at the entrance, one may use either the water pipes along the inner side to pour pupae, or may have an additional pipe going in parallel to that dispensing pupae along the entire length of the drum.

The collection containers or trays may be moved to other stations, say for rearing the insects after collection.

Reference is now made to FIG. 42 which illustrates a way in which the disc shapes may be varied in order to produce openings with similar effect. Instead of having a convex shape on both sides of the discs, the structure can be made of a set of discs, where only a single disc 310 of each pair that creates the gap has a convex shape and one disc 312 of each pair is flat.

Additional Venturi water pumps may be introduced at the male pupae tray, guiding the material from the male tray back to the Mixed Insect Tray for repeated sorting. As such, the male only tray may include larvae that didn't fall through at the first stage (if the discs were clogged for example, or the length of the larvae area was too short in contrast to the amount of insect material that was introduced or the speed of rotation of the drum, propagating the material too fast). Thus male plus larva are then guided again and transferred towards the mixed tray, to be sorted again, with increased chances that more larvae will be sorted out.

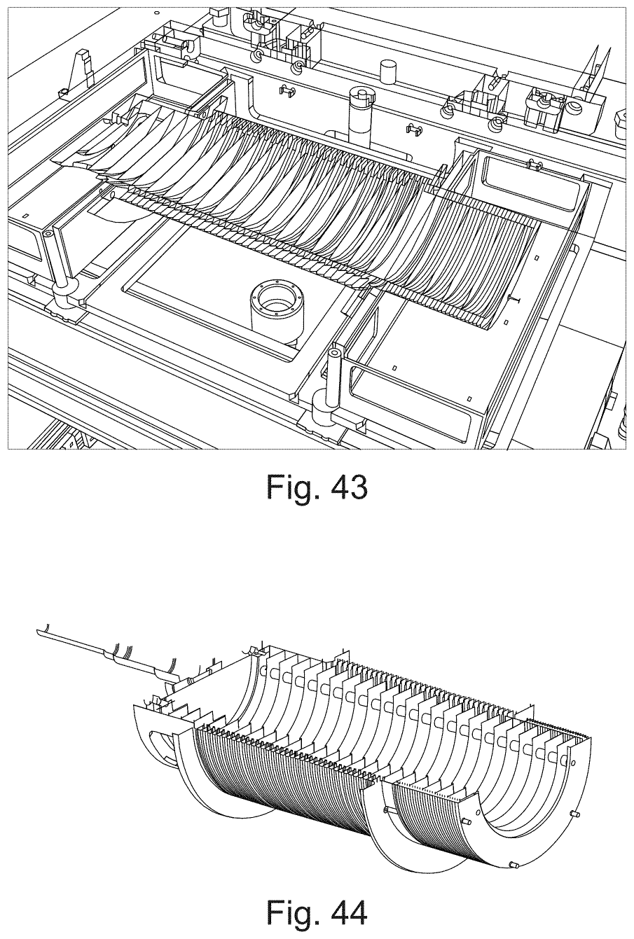

Reference is now made to FIGS. 43 and 44, which show an embodiment wherein the discs are fixed semicircles and do not rotate. Instead, the up and down movement causes the sorting to happen, and the helical screw or spiral structure creates the propagation along the drum.

Unclogging of insects in this case is solved by mechanical elements that can be inserted from either below or above to push the clogged material back into the general flow.

In the main embodiment the helical screw is an external element which for simplicity is then inserted into the drum structure from one end to the other. In another embodiment the screw element is manufactured together with the discs and is part of the entire structure.

In another embodiment the knife element for unclogging the male pupae section may also extend along the larvae area.

In another embodiment the larvae discs have the same shape as the pupae part discs, only with different spacing.

While there is an advantage of having three sections viz female pupa, male pupa and larvae, it is possible the machine may include only include two sections, for female and male pupae.

The separation of both larvae and male at the same machine along the drum is possible because the helical screw may first sort the insects between larva and non-larvae, only after which they continue traveling towards the female and male areas.

In another embodiment, the internal pipe that injects water along the entire drum, is used to convey insect material, that is male and female pupae along the entire internal drum section. The pupae are then flushed out of the pipe, and fall down towards the discs. As the discs move upwards, the males are flushed down, while the females are not, and are then propagated along the internal structure all the way towards the female location.

The main embodiment has the advantage, that as the insect material is injected at the entrance, it has multiple chances to be sorted, one for each gap passed.

In another embodiment, the insect material is propagated along the discs by means of rotating blades at either side of the drum, which pushes or pulls the water. The blades may also move the water forward towards the female section. However, using a spiral screw like element enables a more slow and controlled motion of the materials, enabling it repeated chances for being sorted along the entire structure, rather than being moved in large distances each time the blades push the water.

In another embodiment, moving of the water with the insects is achieved by tilting the entire drum up and down. As the drum goes down, water enters in between the discs, and insects may be moved. Some small objects which were not in the right orientation now have the chance of being sorted again. After rotating and moving the drum is lifted and the process repeats. embodiment.

In an embodiment, the drum is tilted up and down to provide flow, without any rotation. As a possibility, the drum is then rotated just for cleaning, for example as more and more females start clogging up the gaps.

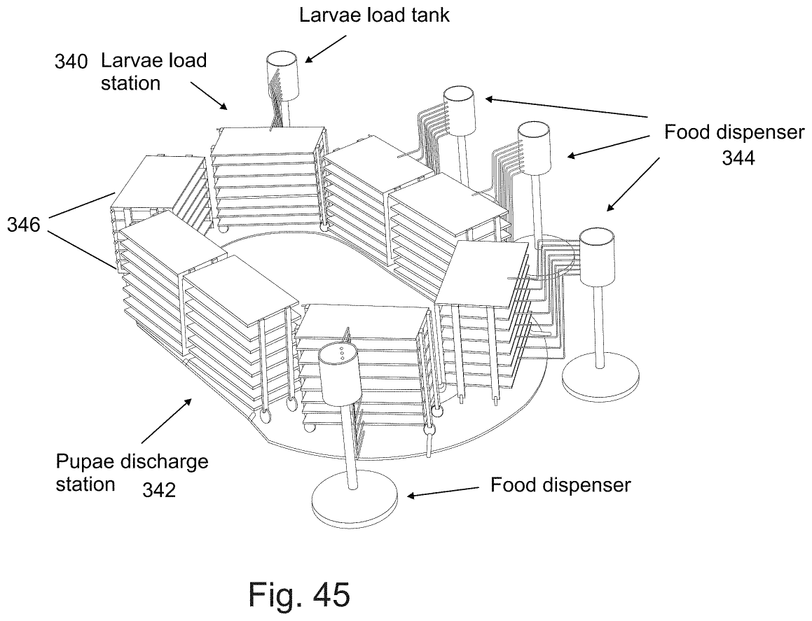

In the above, the process of sorting pupae and separating pupae and larvae where considered. Reference is now made to FIG. 45 which relates to a rearing process that is cyclic and works with the sorting process. A group of movable trays of larvae, are conveyed in synchronization with their pupation time, so that at a certain position, referred to herein as the Larva loading station 340, larvae are loaded into the larvae water trays. Once all larvae are loaded, then at a later time, say on the following day, the trays are conveyed away and replaced with empty trays. Within a few days, corresponding to the number of days it takes for pupation, the trays reach the pupation station 342, at which time valves discharging pupae from the larvae trays towards pupa trays. The rack holding the trays may move in discrete moves, for example one move per day to move between feeding stations 344, or it can continuously move at very low speed towards the next feeding station. The movement of the larvae trays, say as a rack 346 or a group of larvae trays, is in a cyclic manner, having an entry point for larvae loading, and a discharging point for discharging the larvae towards the pupae containers as preferably two different stations within the cyclic line.

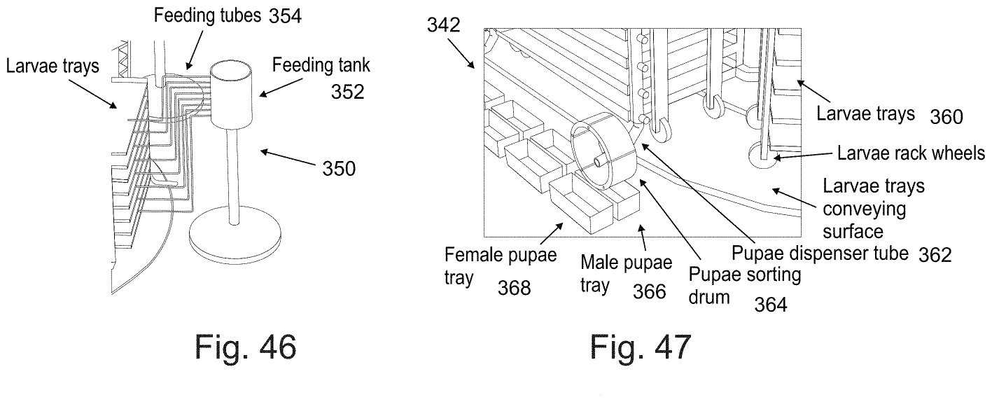

The mosquitoes are propagated in a cyclic manner between stations corresponding to their development stage. Each day, a different amount of food may be dispensed to the larvae depending on the development stage, while at the station corresponding to their expected pupal stage, there is no feeding since pupae do not require food. FIG. 46 shows a typical feeding station 350 along the production cycle line in which a tank 352 distributes food via feeding tubes 354 to the larva trays. The food dispenser may comprise a set of pipes as shown, each going directly towards a location where they dispense the food into the larvae tray. Alternatively, there can be a single feeding pipe, being able to move up and down and feed each time a different larvae tray. Such movement can be implemented by commercial products such a linear rail structure with positioning sensor providing feedback for the system control where the feeding pipe is positioned at every moment.

Reference is now made to FIG. 47, which is a detail of Pupa discharge station 342. At pupa discharge station 342 the trays are automatically drained, and pupae are discharged as the trays are emptied. The process involves pouring the pupae and any remaining larvae towards pupa containers. As shown in FIG. 47, the pupae exit from larva trays 360 via dispensing pipe 362 into drum 364, which is the sorting drum described hereinabove. The pupae are thus sorted into a male tray 366 and a female tray 368 and optionally a larva tray (not shown).

Over a 24 hour period, the entire rack may be moved in cyclic manner. The rack may move on rails. An empty rack comes from the other side to be refilled.

In such way, the racks are moved until they reach a pupa discharge station 342. This location corresponds to the time at which typically more than half of the larvae are transformed into pupae.

At station 342, a trigger may be initiated, the larva trays 360 are drained, preferably into pupae containers, as discussed.

The pupa containers may also move in cyclic manner.

The larvae rack holding the group of larvae trays stops at the pupae dispensing position. Any system may be used for detecting the position, such as an rf-id sensor, a touch sensor, an IR sensor etc.

The pupae conveying system, may convey an empty container and position it adjacent to the dispensing pipe to collect the incoming pupae from the larvae trays.

The pupae container may be smaller in its dimensions (as least the X-Y face surface) than the larvae tray, since pupae can be stored at much higher density than larvae.

The larvae trays 360 may be emptied one after the other by means of opening valves which connect each larvae tray with pipe 362. Pipe 362 may be connected to all the trays and goes down towards the pupa tray dispensing point.

Controller 40, for example software encoded, may open each valve to empty each tray one after the other. The trigger to start opening the next valve may be a counter (timer), or any other sensor that takes feedback from the mechanical system. The controller is then set to open the next valve, and as necessary potentially to close the current open valve. When the pupa container is filled with sufficient material, water with pupae, the pupa container conveying system may command the conveying system to propagate the container forward and bring in another empty container to start filling it and repeat the process.

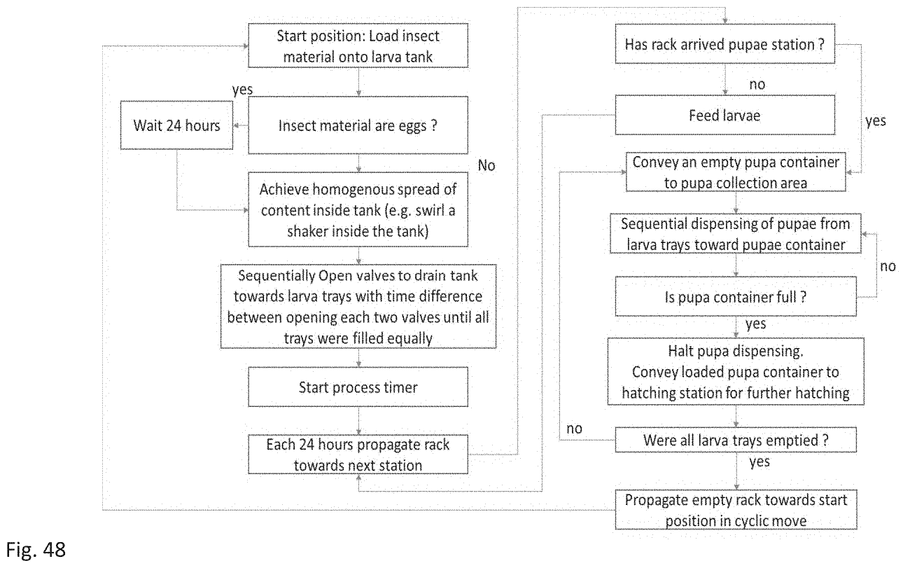

Reference is now made to FIG. 48 the process flow is shown in graphical format. detailed process flow is described in the following diagrams, excluding the pre-sorting process for the male-female pupae utilizing, that is not including the rotating drum.

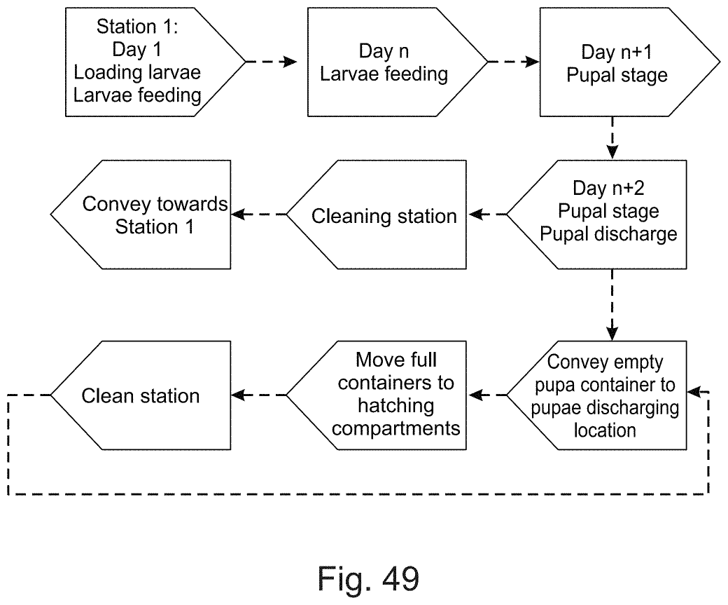

Reference is now made to FIG. 49 which illustrates the process according to a daily schedule.

Regarding FIG. 49, a cleaning station is not mandatory, and cleaning may either be a manual operation or automatic. Cleaning may typically involve flushing water into the trays, and using the same pupae discharging mechanism to flush down the water to finish the cleaning operation.

Although the trays are shown as a group of rectangular plates, they can also be round or half tubes, or any other shape for storage of both liquid and larvae.

The trays may not need to be taken in and out of the racks, and are filled and discharged directly from their location.

The production line is cyclic and may include designated entry loading and unloading stations.

The production line may include a single cycle of the larvae racks, with start point (loading larvae) and discharge station (discharge pupae) and the handling and propagation of the pupae container can be handled manually as discussed.

The discharge of the pupae at the discharge position may be directed onto release boxes. In such a variant the larvae circular production line interfaces with a linear production line which conveys empty pupa containers towards the pupa discharge position where they are loaded, and then conveys the loaded containers to a logistic handling position, where an employee may take away the loaded container with pupae for storage.

In another variant the employee may take away the pupa container once it is filled, based on visual and/or an audible feedback notifying that it is full, and once the loaded container is taken, the next empty container is brought in place, and interfaces with the discharge pipe for discharge of the pupae from the larvae rack using the manipulation of the valves as described above.

When more than one larva tray is positioned at the same height, then when opening the valves to discharge the tray content, it is possible to open more than a single tray at once.

The above solution describes a system and a method in which two circles, one for rearing larva and the other for maintaining the pupae, feed each other.

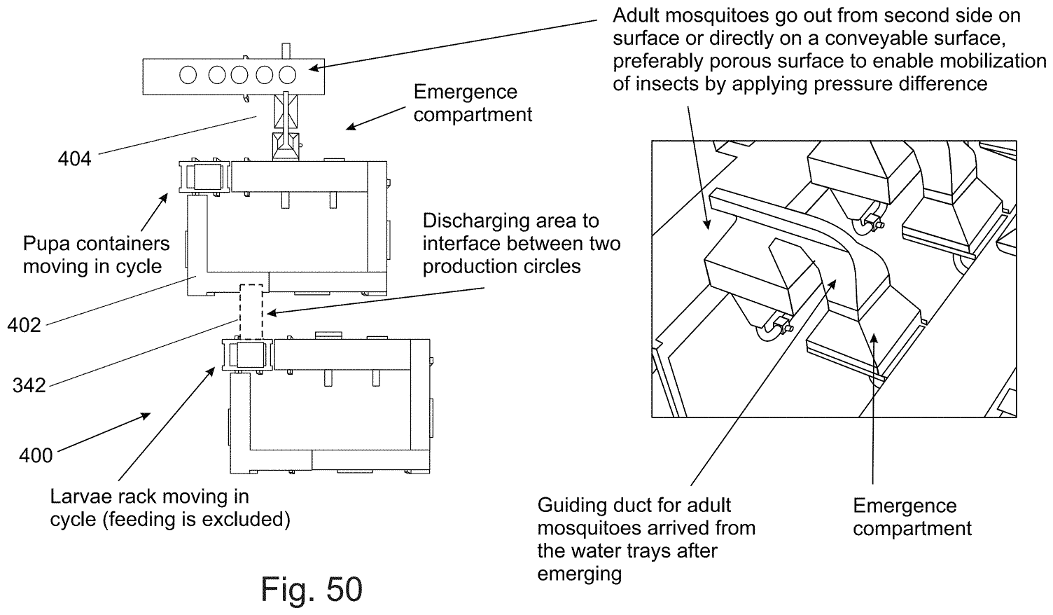

Reference is now made to FIG. 50, which is a view from above of two production circles interfacing each other, wherein a larva circle 400 feeds a pupae circle 402, when pupae material flows from one circle to the other at the pupa discharge station 342. Emergence (hatching) compartment 404 is where adult mosquitoes emerge from the pupae.

Once the mosquitoes emerge from their pupae, they are guided towards the collection area, where they can then be loaded as adults (either automatically by means of a blower for example or manually) into release boxes.

The embodiment of FIG. 50 describes a continuous larva rearing process; with single inlet position within that circle; single outlet position and an interface with a second circular rearing line for the manipulation of the pupae containers.

The entire rearing process of the mosquito from the egg (or larvae) stage all the way until they are adults, is achieved using the double circle cyclic production line with pupa loading line of FIG. 50.

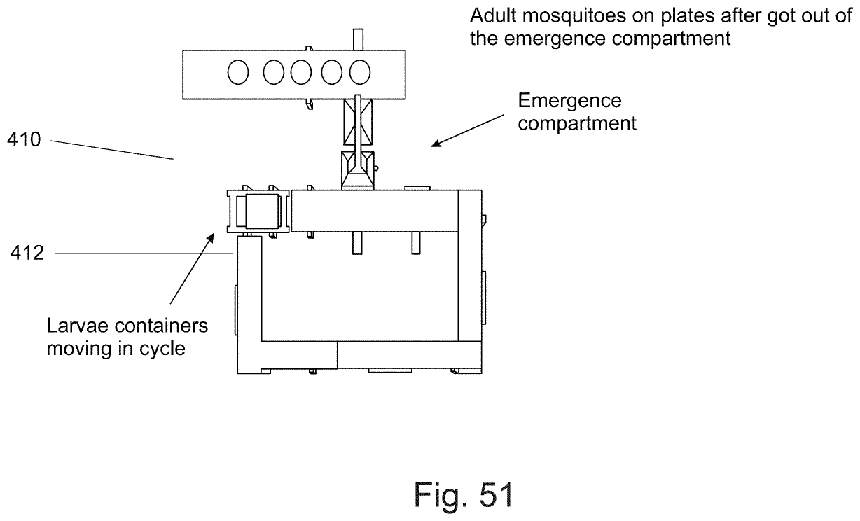

Referring now to FIG. 51, in an embodiment, there is no pupae dispensing station, and instead there is a hatching station 410 as part of a single cycle 412.

In that case the entire larvae rack enters into the hatching station as one of the stations in the cyclic production line, and air flow may guide the hatching mosquitoes towards a collection area which can be fixed or conveyable. The emerging mosquitoes are thus guided from the larvae trays, at which point in time they are almost all pupae and not larvae, to the collection area and may be immobilized by means of applying either pressure difference to the collection surface, which is preferably porous to enable pressure difference to provide the required effect on the misquotes and make them stay fixed on the surface, or cold air or anesthetizing gas (e.g. CO2). Once immobilized they can more easily managed (e.g. collected into release boxes).

In an embodiment, the larvae trays and the circular design of the line is such that the trays enter the hatching compartment on the day, or the day after, the larvae hatch and are transformed into pupae, and so for the next 1-2 days as the line stops, the pupa turn into adults, and then inside the hatching compartment the adults are guided by air flow towards a collection area. The collection area may be conveyable or fixed, and may be porous to enable the air to move through. Once the adults arrive at the collection area, they are moved away for packaging into release boxes. In an embodiment, the adults are guided directly into release boxes, and the release boxes are conveyed away once they were filled with sufficient number of insects. As above, a sensor such as camera or light detector may be located to count the number of insects that arrive in the duct leading towards the release box, and, once the box is full, a new empty box may be placed at the collection area. If mosquitoes are required not to move, that is they are immobilized, then a pressure difference applied at the collection area on which mosquitoes are located, may cause the mosquitoes at the collection area to be stationary or relative stationary, to an extend sufficient for logistic handling.

In an embodiment, the pupa containers may serve as release boxes for release of sterile mosquitoes, and thus may discharge the pupae into pupae containers which are release boxes. Such empty release boxes may be conveyed along a conveying system to be automatically loaded with the pupae.

When pouring the material directly into the pupae release box, an amount of pupae poured outside of the larvae tray may be estimated.

There may be a single queue of larva trays in a cycle with a single output to pupae, or the trays may be in racks.

Two queues of containers of different types may thus be propagated (in a cyclic pattern) in sync with each other. One queue is a continuous cyclic line of larva containers as discussed and the second queue is that of the pupa containers, which are propagated in sync so to park an empty (or not yet filled) pupa container at the larvae dispensing station.

Each tray may have its temperature separately controlled by heating the water therein.

The terms “comprises”, “comprising”, “includes”, “including”, “having” and their conjugates mean “including but not limited to”.

The term “consisting of” means “including and limited to”.

As used herein, the singular form “a”, “an” and “the” include plural references unless the context clearly dictates otherwise.

It is appreciated that certain features of the invention, which are, for clarity, described in the context of separate embodiments, may also be provided in combination in a single embodiment and the present description is to be construed as if such embodiments are explicitly set forth herein. Conversely, various features of the invention, which are, for brevity, described in the context of a single embodiment, may also be provided separately or in any suitable subcombination or may be suitable as a modification for any other described embodiment of the invention and the present description is to be construed as if such separate embodiments, subcombinations and modified embodiments are explicitly set forth herein. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

In addition, any priority document(s) of this application is/are hereby incorporated herein by reference in its/their entirety.

1. The device of claim 2, the drum being at a tilt such that said first end is higher than said second end to enable flow from said first end to said second end, wherein said openings comprise gaps between discs, a size of each opening being defined between respective discs, the discs having inner and outer edges on either side of an interior respectively, and wherein at least some of said discs have a longitudinal cross section of which at least one side is convex at said inner and outer edges and flat along said interior.

2. A device for sorting insects into two or more classes based on size, the device comprising a drum, the drum having an interior being closed circumferentially and openings defined along said circumference, and said insects being introduced to said drum at a first end, the openings being sized to allow a first class of said insects to exit said drum through said circumference while said second class is retained to travel to a second end, thereby sorting said insects into said at least two classes, the drum further comprising a helix shape in said interior to cause flow through said interior as said helix is rotated, wherein said openings comprise gaps between discs, a size of each opening being defined between respective discs, the discs having inner and outer edges on either side of an interior respectively, and wherein at least some of said discs have a longitudinal cross section of which at least one side is convex at said inner and outer edges and flat along said interior.

3. The device of claim 2, the drum being mounted and motorized for rotating, the drum being located below water outlets, the outlets being configured to pour water on said openings as said openings rotate past said water outlets to flush out insects stuck in said openings, the openings comprising gaps between discs the discs having an inner extent, the drum being mounted for raising and lowering with respect to an external water level between a first position wherein inner extents of said discs at a lower side of said drum are submerged and an second position in which at least the drum internal surface is above water level.

4. The device of claim 2, the drum being located against a bank of mechanical protrusions, the mechanical protrusions being configured to extend into said openings as said openings rotate past said mechanical protrusions to clean out insects stuck in said openings.

5. The device of claim 2, the drum being mounted and motorized for rotating, the openings comprising gaps between a plurality of discs, the discs each having a respective inner radius, the discs comprising a first class of discs interspersed with a second class of discs, the inner radius of discs in the first class of discs being smaller than the inner radius of discs in the second class of discs, said discs of the first class of discs thereby extending further into said interior than said discs of said second class of discs.

6. The device of claim 2, comprising an internal pipe for injecting water into said interior to flush said openings.

7. The device of claim 2, the drum being mounted and motorized for rotating, the device comprising at least one collection container underneath said drum to collect said insects exiting through said circumference and a controller configured to exchange said collection container when full of insects.

8. The device of claim 7, comprising a funnel underneath said drum, the funnel leading to said collection container, to collect said insects exiting through said circumference, and an indicator located at said collection container to provide an indication when said collection chamber contains a pre-defined number of insects.

9. A device for sorting insects into two or more classes based on size, the device comprising a drum, the drum having an interior being closed circumferentially and openings defined along said circumference, and said insects being introduced to said drum at a first end, the openings being sized to allow a first class of said insects to exit said drum through said circumference while said second class is retained to travel to a second end, thereby sorting said insects into said at least two classes, the openings comprising gaps between discs the discs having an inner extent, the drum being mounted for raising and lowering with respect to an external water level between a first position wherein inner extents of said discs at a lower side of said drum are submerged and a second position in which at least the drum internal surface is above water level.

10. The device of claim 9, the drum comprising a helical screw rotatably mounted to rotate relative to the drum to propel insects and water from said first end to said second end.

11. The device of claim 2, the circumference being divided into at least two sections, each section having a spacing sized for a predetermined and respectively different class of insects and at least one opening sized for said respectively different class of insect, a second end of said drum being open for exit by a third class of insects, said third class of insects being unsorted by said two sections.

12. The device of claim 2, being configured for flow of water through said drum from said first end to said second end, to allow said third class to flow out of said second end.

13. The device of claim 2, comprising a rotating screw inserted into the drum, or comprising a helical shape built onto an interior of said circumference.

14. (canceled)

15. The device of claim 2, wherein the at least two classes are male and female pupae, the male and female pupae being of different sizes and said openings being of a size selected to allow male pupae to pass through but to inhibit female pupae.

16. The device of claim 15, wherein the at least two classes are three classes, larvae, male pupae and female pupae, the drum comprising two sections, a first section with openings sized for larvae, and a second section with openings sized for male pupae, or wherein said at least two classes are four classes, larvae, small male pupae, large male pupae and female pupae, the drum comprising three sections, a first section with openings sized for larvae, a second section with openings sized for small male pupae and a third section with openings sized for large male pupae.

17. (canceled)

18. The device of claim 16, comprising an input at said first end, the input for receiving a mixture of water, larvae and male and female pupae, the input comprising a pourer for pouring said mixture into said drum.

19. The device of claim 18, wherein the pourer is configured to pour out said mixture into said interior in a plane of said first end or over a length of said drum.

20. (canceled)

21. The device of claim 2, wherein said drum is a stationary mounted half drum, or wherein said drum is rotatably mounted.

22. (canceled)

23. The device of claim 4 wherein said mechanical projections are mounted to a frame via a tensioned mount.

24. The device of claim 2, wherein said openings comprise cuts in a cylinder.

25. The device of claim 2, wherein said openings comprise gaps between discs, a size of each opening being defined by a spacer between respective discs.

26. The device of claim 25, wherein at least some of said discs have a longitudinal cross section comprising convex outer edges and flat interiors, or wherein at least some of said discs have a longitudinal cross section that is flat.

27. (canceled)

28. The device of claim 2, comprising drainage collection underneath said drum and a pump to recirculate water from said drainage for reuse in said drum.

29. The device of claim 1, the drum being mounted such that the angle of said tilt is adjustable or wherein the rate of pouring is adjustable.

30-35. (canceled)

36. A device for sorting insects into two or more classes based on size, the device having openings, and said insects being introduced to said device at a first end, the openings being sized to allow a first class of said insects to exit said device while said second class is retained to travel to a second end, thereby sorting said insects into said at least two classes, the openings comprising discs and gaps between said discs, the discs having an inner extent, the device being mounted for raising and lowering with respect to an external water level between a first position wherein inner extents of said discs at a lower side of said device are submerged and a second position in which at least an internal surface is above water level.
